(19) United States
(12) Patent Application Publication
(10) Pub. No.: US 2002/0127238 A1
(43) Pub. Date: Sep. 12, 2002

(54) HIV-1 vaccines and screening methods therefor

(76) Inventors: Leonidas Stamatatos, Seattle, WA (US); Susan W. Barnett, San Francisco, CA (US); Indresh K. Srivastava, Benicia, CA (US)

Correspondence Address: KLAUBER & JACKSON, 411 HACKENSACK AVENUE, HACKENSACK, NJ, 07601

(21) Appl. No.: 09/891,609

(22) Filed: Jun. 26, 2001

Related U.S. Application Data
(60) Provisional application No. 60/214,608, filed on Jun. 27, 2000.

(51) Int. Cl. A61K 48/00; A61K 39/21

(52) U.S. Cl. 424/188.1; 514/44

(57) ABSTRACT

Methods for immunizing, and immunogen pharmaceutical compositions for eliciting a heterologous immune response to HIV-1 in an animal, preferably a human, are provided, utilizing a modified HIV-1 envelope protein or fragment or DNA encoding a modified HIV-1 envelope protein or fragment, the modified protein having a HIV-1 envelope protein V2 region deletion. A humoral response against heterologous HIV-1 strains is achieved.

### CROSS-REFERENCE TO RELATED APPLICATION

Priority under 35 U.S.C. §119(e) is claimed to Provisional Application Serial No. 60/214,608, filed Jun. 27, 2000, and which is incorporated herein by reference in its entirety.

### RESEARCH SUPPORT

The research leading to the present invention was supported in part by the Public Health Service, National Institutes of Health grants AI47708-01 and AI44309-01. The government may have certain rights in the present invention.

### BACKGROUND OF THE INVENTION

DNA immunization stimulates both the cellular and humoral arms of the immune system (Liu, M. A., Y. Yasutomi, M.-E. Davis, H. C. Perry, D. C. Freed, N. L. Letvin, and J. W. Shiver. 1996. Vaccination of mice and nonhuman primates using HIV-gene-gun-containing DNA, vol. 48. Karger, S, Basel; Shiver, J. W., M.-E. Davies, H. C. Perry, D. C. Freed, and M. A. Liu. 1996. Humoral and cellular immunities elicited by HIV-1 DNA vaccination. J. Pharm. Sci. 85:1317-1324; Shiver, J. W., H. C. Perry, M.-E. Davies, D. C. Freed, and M. A. Liu. 1995. Cytotoxic T lymphocyte and helper T cell responses following HIV polynucleotide vaccination. DNA Vaccines. 772:198-208; Shiver, J. W., J. B. Ulmer, J. J. Donnely, and M. A. Liu. 1996. Humoral and cellular immunities elicited by DNA vaccines: Application to the human immunodeficiency virus and influenza. Adv. Drug Del. Rev. 21:19-31-18) and elicits immune responses capable of preventing infection of animals by slowly replicating viruses, such as HIV-1 in chimpanzees (Boyer, J. D., K. E. Ugen, B. Wang, M. Agadjanyan, L. Gilbet, M. L. Bagarazzi, M. Chattergoon, P. Frost, A. Javadian, W. V. Williams, Y. Refaeli, R. B. Ciccarelli, D. McCallus, L. Coney, and D. B. Weiner. 1997. Protection of chimpanzees from high-dose heterologous HIV-1 challenge by DNA vaccination. Nature Med. 3:526-532). However, when the challenge virus replicates efficiently in the host, such as SIV or SHIV in macaques, the DNA-elicited immune responses offer only partial protection (Boyer, J. D., B. Wang, K. E. Ugen, M. Agadjanyan, A. Javadian, P. Frost, K. Dang, R. A. Carrano, R. Ciccarelli, L. Coney, W. V. Williams, and D. B. Weiner. 1996. In vivo protective anti-HIV immune responses in non-human primates through DNA immunization. J. Med. Primatol. 25:242-250; Lu, S., J. Arthos, D. C. Montefiori, Y. Yasutomi, K. Manson, F. Mustafa, E. Johnson, J. C. Santoro, J. Wissink, J. I. Mullins, J. R. Haynes, N. L. Letvin, M. Wyand, and H. L. Robinson. 1996. Simian immunodeficiency virus DNA vaccine trial in macaques. J. Virol. 70:3978-3991; Robinson, H. L., D. C. Montefiori, R. P. Johnson, K. H. Manson, M. L. Kalish, J. D. Lifson, T. A. Rizvi, S. Lu, S. L. Hu, G. P. Mazzara, D. L. Panicali, J. G. Herndon, R. Glickman, M. A. Candido, S. L. Lydy, M. S. Wyand, and H. M. McClure. 1999. Neutralizing antibody-independent containment of immunodeficiency virus challenges by DNA priming and recombinant pox virus booster immunizations. Nature Medicine. 5:526-34). To increase the potency of these responses, especially the development of high anti-HIV/SIV envelope antibody titers, follow-up administration of soluble viral envelope proteins, viral particles or recombinant vaccinia-based viruses expressing the HIV/SIV envelope is required (Agadjanyan, M. G., N. N. Trivedi, S. Kudchodkar, M. Bennett, W. Levine, A. Lin, J. Boyer, D. Levy, K. E. Ugen, J. J. Kim, and D. B. Weiner. 1997. An HIV type 2 DNA vaccine induces cross-reactive immune responses against HIV type 2 and SIV. AIDS Res. Hum. Retroviruses. 13:1561-1572; Barnett, S. W., J. M. Klinger, B. Doe, C. M. Walker, L. Hansen, A. M. Duliege, and F. M. Sinangil. 1998. Prime-boost immunization strategies against HIV. AIDS Res. Hum. Retroviruses. 14 Suppl 3:S299-309; Letvin, N. L., D. C. Montefiori, Y. Yasutomi, H. C. Perry, M.-E. Davies, C. Lekutis, M. Alroy, D. L. Freed, C. I. Lord, L. K. Handt, M. A. Liu, and J. W. Shiver. 1997. Potent protective anti-HIV immune responses generated by bimodal HIV envelope DNA plus protein vaccination. Proc. Natl. Acad. Sci. 94:9378-9383; Richmond, J. F., S. Lu, J. C. Santoro, J. Weng, S. L. Hu, D. C. Montefiori, and H. L. Robinson. 1998. Studies of the neutralizing activity and avidity of anti-human immunodeficiency virus type 1 Env antibody elicited by DNA priming and protein boosting. J Virol. 72:9092-100; Richmond, J. F. L., F. Mustafa, S. Lu, J. C. Santoro, J. Weng, M. O° Connell, E. M. Fenyo, J. L. Hurwitz, D. C. Montefiori, and H. L. Robinson. 1997. Screening of HIV-1 Env glycoproteins for the ability to raise neutralizing antibody using DNA immunization and recombinant vaccinia virus boosting. Virology. 230:265-274; Robinson, H. L., D. C. Montefiori, R. P. Johnson, K. H. Manson, M. L. Kalish, J. D. Lifson, T. A. Rizvi, S. Lu, S. L. Hu, G. P. Mazzara, D. L. Panicali, J. G. Herndon, R. Glickman, M. A. Candido, S. L. Lydy, M. S. Wyand, and H. M. McClure. 1999. Neutralizing antibody-independent containment of immunodeficiency virus challenges by DNA priming and recombinant pox virus booster immunizations. Nature Medicine. 5:526-34). This bimodal method of immunization elicits responses capable of protecting Rhesus macaques (Rh) from infection by SHIV (Letvin, N. L., D. C. Montefiori, Y. Yasutomi, H. C. Perry, M.-E. Davies, C. Lekutis, M. Alroy, D. L. Freed, C. I. Lord, L. K. Handt, M. A. Liu, and J. W. Shiver. 1997. Potent protective anti-HIV immune responses generated by bimodal HIV envelope DNA plus protein vaccination. Proc. Natl. Acad. Sci. 94:9378-9383; Robinson, H. L., D. C. Montefiori, R. P. Johnson, K. H. Manson, M. L. Kalish, J. D. Lifson, T. A. Rizvi, S. Lu, S. L. Hu, G. P. Mazzara, D. L. Panicali, J. G. Herndon, R. Glickman, M. A. Candido, S. L. Lydy, M. S. Wyand, and H. M. McClure. 1999. Neutralizing antibody-independent containment of immunodeficiency virus challenges by DNA priming and recombinant pox virus booster immunizations. Nature Medicine. 5:526-34). However, because during the above method of vaccination both cellular as well as humoral anti-viral responses were generated, it is unclear whether the recorded protection was mediated by the cellular and/or humoral anti-viral responses elicited during DNA immunization. By evaluating and comparing the respective anti-viral protective roles of these two types of responses, more effective DNA immunization protocols may be developed.

Analysis of the crystal structure of the gp120 HIV envelope subunit indicated that neutralization epitopes are primarily clustered in one face of this protein, which is naturally occluded within the oligomeric envelope form, i.e., that present on the surface of virions and infected cells (Kwong, P. D., R. Wyatt, J. Robinson, R. W. Sweet, J. Sodroski, and W. A. Hendrickson. 1998. Structure of an HIV gp120 envelope glycoprotein in complex with the CD4 receptor and a neutralizing human antibody. Nature (London) 393:648-659; Wyatt, R., P. D. Kwong, E. Desjardins, R. W. Sweet, J. Robinson, W. A. Hendrickson, and J. G. Sodroski. 1998. The antigenic structure of the HIV gp120 envelope glycoprotein. Nature (London) 393:705-711). These structural observations are supported by numerous immunochemical and virological studies (Bou-Habib, D. C., G. Roderiquez, T. Oravesz, P. W. Berman, P. Lusso, and M. A. Norcross. 1994. Cryptic nature of envelope V3 region epitopes protects primary monocytotropic human immunodeficiency virus type 1 from antibody neutralization. J. Virol. 68:6006-6013; Moore, J. P., J. A. McKeating, Y. Huang, A. Askenazi, and D. D. Ho. 1992. Virions of primary human immunodeficiency virus type 1 isolates resistant to soluble CD4 (sCD4) neutralization differ in sCD4 binding and glycoprotein gp120 retention from sCD4-sensitive isolates. J. Virol. 66:235-243; Reitter, J. N., R. E. Means, and R. C. Desrosiers. 1998. A role for carbohydrates in immune evasion in AIDS. Nat. Med. 4:679-684; Sattentau, Q. J., and J. P. Moore. 1991. Conformational changes induced in the human immunodeficiency virus envelope glycoprotein by soluble CD4 binding. J. Exp. Med. 174:407-415; Sattentau, Q. J., J. P. Moore, F. Vignaux, F. Traincard, and P. Poignard. 1993. Conformational changes induced in the envelope glycoproteins of the human and simian immunodeficiency viruses by soluble receptor binding. J. Virol. 67:7383-7393; Stamatatos, L., and C. Cheng-Mayer. 1995. Structural modulations of the envelope gp120 glycoprotein of human immunodeficiency virus type 1 upon oligomerization and differential V3 loop epitope exposure of isolates displaying distinct tropism upon virion-soluble receptor binding. J. Virol. 69:6191-6198; Sullivan, N., Y. Sun, J. Li, W. Hofmann, and J. Sodroski. 1995. Replicative function and neutralization sensitivity of envelope glycoproteins from primary and T-cell line-passaged human immunodeficiency virus type 1 isolates. J. Virol. 69:4413-4422; Wyatt, R., J. Moore, M. Accola, E. Desjardin, J. Robinson, and J. Sodroski. 1995. Involvement of the V1/V2 variable loop structure in the exposure of human immunodeficiency virus type 1 gp120 epitopes induced by receptor binding. J. Virol. 69:5723-5733; Wyatt, R., N. Sullivan, M. Thali, H. Repke, D. Ho, J. Robinson, M. Posner, and J. Sodroski. 1993. Functional and immunologic characterization of human immunodeficiency virus type 1 envelope glycoproteins containing deletions of the major variable regions. J. Virol. 67:4557-4565).

It is towards the enhancement of effective vaccination against HIV-1 that the present invention is directed.

The citation of any reference herein should not be deemed as an admission that such reference is available as prior art to the instant invention.

### SUMMARY OF THE INVENTION

In accordance with the present invention, a method is provided for eliciting a heterologous immune response to HIV-1 in an animal by immunizing the animal with an immunogen comprising at least one modified HIV-1 envelope protein or fragment thereof, or DNA or virus encoding said at least one modified HIV-1 envelope protein or fragment thereof, or any combination thereof, the modified envelope protein having a HIV-1 envelope protein V2 region deletion. The modified HIV-1 envelope protein may be a recombinant protein of fragment thereof expressed in mammalian cells. Preferably, the modified HIV-1 envelope protein or fragment thereof is glycosylated. The immunized animal exhibits an immune response to at least one HIV-1 strain other than that of the immunogen. In a preferred embodiment, the immune response comprises a humoral response. In a more preferred embodiment, the humoral response includes neutralizing antibodies, and most preferred, protective antibodies. Preferably, the animal is a human.

In a non-limiting example, the immunogen comprises a modified HIV-1 envelope protein or fragment thereof from a clade-B HIV-1 strain, or DNA or a virus encoding a modified HIV-1 envelope protein of fragment thereof from a clade-B HIV-1 strain. In a preferred embodiment, the HIV-strain is SF162. By way of example, the modified HIV-1 envelope protein or fragment thereof is SEQ ID No:2 or SEQ ID No:4; and a DNA encoding the at least one modified HIV-1 envelope protein or fragment thereof is SEQ ID No:1 or SEQ ID No:3.

In another broad aspect of the invention, a vaccine pharmaceutical composition is provided for immunizing an animal against HIV-1 virus, the vaccine pharmaceutical composition comprising an effective heterologous immune-response-eliciting amount of at least one modified HIV-1 envelope protein or fragment thereof, DNA or virus encoding the at least one modified HIV-1 envelope protein or fragment thereof, or a combination thereof, the modified envelope protein or fragment thereof having an HIV-1 envelope protein V2 region deletion; and a pharmaceutically-acceptable carrier or excipient. The modified HIV-1 envelope protein or fragment thereof may be expressed in a mammalian cell. It may be glycosylated. In one embodiment, the modified HIV-1 envelope protein or fragment thereof is from a clade-B HIV-1 strain. In a preferred embodiment, the HIV-1 strain is SF162. By way of non-limiting examples, the modified HIV-1 envelope protein or fragment thereof is SEQ ID No:2 or SEQ ID No:4; and a DNA encoding said at least one modified HIV-1 envelope protein or fragment thereof is SEQ ID No:1 or SEQ ID No:3. Immunization or vaccination of an animal with the foregoing vaccine pharmaceutical composition elicits a heterologous immune response to HIV-1. The response comprises a humoral response. In one embodiment, the humoral response comprises neutralizing antibodies. In a preferred embodiment, the elicited antibodies are protective.

The invention is also directed to a method for assessing whether a compound is capable of generating at least neutralizing antibodies in an animal against at least one heterologous strain of HIV-1 comprising the steps of immunizing the animal with the compound, depleting the animal of its CD8+ cells, and screening the animal for the presence of neutralizing antibodies, or preferably protecting antibodies, to at least one heterologous strain of HIV-1. In one embodiment, the depleting is carried out by administering to said animal anti-CD8 monoclonal antibodies. The compound may be an HIV-derived polypeptide of fragment thereof or DNA or virus encoding the peptide or fragment thereof; and the immunogen comprise a viral or DNA vaccine, a protein, or a combination thereof. Preferably, the protective antibodies are neutralizing antibodies, and most preferably protective antibodies. For detecting protective antibodies, the animal is injectable with the wild-type HIV-1 or SHIV strain, or one capable of developing a protective antibody response to wild-type HIV-1 or SHIV-1.

The invention is further directed to a method for making a protein, protein fragment, DNA or viral immunogen encoding the protein or protein fragment, as described above. Preferably, the protein immunogen is expressed in a mammalian cell and is therefore glycosylated.

These and other aspects of the present invention will be better appreciated by reference to the following drawings and Detailed Description.

### BRIEF DESCRIPTION OF THE DRAWINGS

FIG. 1 depicts the generation of anti-HIV envelope binding antibodies during immunization. The envelope-specific titers of binding antibodies in animals J408 and H445 throughout the immunization schedule were determined against the vaccine, i.e., the purified oligomeric SF162ΔV2 gp140 protein. Dashed lines indicate the time of immunization and the arrow indicates the time of viral-challenge.

FIG. 2 depicts the generation of HIV-1 neutralizing antibodies. The presence of neutralizing antibodies against the homologous SF162ΔV2 virus and the parental SF162 viruses was determined at various time points during the immunization schedule: ∘: pre-bleeds; ▴: 1 month post the third DNA immunization; ▪: 2 weeks following the first ‘boost’; and ♦: 2 weeks following the second ‘boost’.

FIG. 3 shows the depletion of CD8+ T lymphocytes: CD8+ T lymphocytes were depleted from the vaccinated animals by bolus injection of the anti-CD8 MAb OKT8F (arrows). The numbers of circulating CD4+ (filled symbols), CD8+ T (open symbols) and total CD3+ T lymphocytes (asterisks) from vaccinated and unvaccinated animals was determined in samples collected at various points prior to and following SHIV162P4-challenge (dashed line).

FIG. 4 A-B depicts the viral load and generation anti-HIV envelope antibody titers following SHIV162P4-exposure: (A) The viral load is expressed as RNA copies per ml of plasma. Dashed lines indicate the detection limit of this assay (<500 copies per ml). † The unvaccinated animal AT54 was euthanized 111 days post-challenge following the development of simian AIDS (SAIDS). The arrow indicates the time at which CD8+ cells re-appeared in the periphery of the vaccinated animals. (B) The generation of anti-HIV envelope antibodies following SHIV162P4-challenge was monitored by SF162ΔV2 gp140-based ELISA methodology. The end-point ELISA titers are presented.

FIG. 5 shows the seroconversion of the animals to SIV-gag/pol and HIV env antigens in the vaccinated and unvaccinated macaques.

FIG. 6 depicts the development of antibodies in rabbits: The generation of anti-envelope antibodies was determined by ELISA methodology. Six animals (A1-A6) were immunized with DNA expressing the unmodified SF162gp140 immunogen and six (A7-A12) with DNA expressing the modified ΔV2gp140 immunogen. Titers were determined 2 weeks following each immunization, by ELISA methodology using the oligomeric SF162gp140 and ΔV2gp140 proteins. Dashed lines indicate the time of each immunization.

FIG. 7 A-B depicts neutralization of the SF162ΔV2 and SF162 viruses by rabbit sera: Results from neutralization experiments using sera collected following the third and fifth immunizations against the SF162ΔV2 (A) and SF162 (B) viruses, are presented. Data are representative of at least three independent experiments. The symbols indicate the mean percent neutralization and the standard deviation from triplicate wells. Dashed lines indicate the 50%, 70% and 90% inhibition of infection. Dashed lines and asterisks (controls) are neutralization curves obtained with sera collected from animals that were immunized with the DNA vector alone and are indicative of non-specific neutralization.

FIG. 8 shows the generation of antibodies in Rhesus macaques: The generation of anti-envelope antibodies in animals (J408 and H445) immunized with the modified AV2gp140 immunogen and two animals (P655 and N472) immunized with the unmodified SF162gp140 immunogen, as well as control animals (M844 and H473) immunized with the DNA vector alone, were determined by ELISA methodology using the corresponding protein. Dashed lines indicate the time of immunizations. DNA: The animals received three monthly immunizations with DNA vectors expressing the gp140 form of each immunogen. Control animals received the DNA vector alone. DNA plus protein: The animals received a fourth DNA immunization and at the same time they were immunized with the corresponding CHO-produced oligomeric gp140 proteins, adjuvanted in MF-59C. Control animals received adjuvant alone.

FIG. 9 A-B shows the neutralizing activity of Rhesus macaque sera: The neutralization activity against the SF162 and SF162ΔV2 viruses of sera collected from animals immunized with the modified ΔV2gp140 (A) and the unmodified (B) SF162gp140 immunogens were determined as described in Example 2. Dashed lines indicate the 50%, 70% and 90% inhibition of infection. Results are representative of three to five independent experiments. Data indicate the mean and standard deviation from triplicate wells. Pre-bleeds: sera collected prior to the initiation of vaccination; second DNA and third DNA: sera collected one month following the second and the third DNA administration, respectively; 2 and 4 weeks post boost: sera collected 2 and 4 weeks following the DNA plus protein ‘booster’ immunization, respectively.

FIG. 10 depicts the neutralization of heterologous clade B primary HIV-1 isolates by macaque sera: The neutralization activities of sera collected 2 and 4 weeks following the DNA plus protein ‘booster’ immunization, against heterologous to the vaccine primary HIV-1 isolates, was determined as described in Example 2. Dashed lines indicate 50%, 70% and 90% inhibition of infection. The values represent the specific neutralization, which is defined as the difference between the percent virus neutralization recorded with sera collected following vaccination and that recorded with sera collected prior to the initiation of vaccination. Data points indicate the mean percent specific neutralization from two independent experiments.

FIG. 11 A-B shows the generation of binding and neutralizing antibodies following the second ‘booster’ immunization with the modified ΔV2gp140 protein: (A) The generation of anti-envelope antibodies in two rhesus macaques (J408 and H445) vaccinated with the modified ΔV2gp140 immunogen were determined by ELISA methodology, as described in Example 2. Dashed lines indicate the time of immunizations. DNA: The animals received three monthly immunizations with DNA vectors expressing the gp140 form of this immunogen; DNA plus protein: the animals received a fourth DNA immunization and purified oligomeric ΔV2gp140 protein; and Protein: the animals were immunized with the purified oligomeric ΔV2gp140 protein alone. (B) Neutralization activities against the SF1 62ΔV2 and SF162 isolates of sera following the second ‘boost’ were compared to that of sera collected following the first ‘boost’ (see also FIG. 4). Non-specific neutralization recorded with pre-immunization sera (pre-bleeds) is also shown.

FIG. 12 A-B shows the presence of anti-V3 loop antibodies in sera collected from macaques immunized with the modified ΔV2gp140 immunogen: The development of anti-V3 loop antibodies was determined with the use of an ELISA methodology using the V3 loop peptide derived from the SF162/SF162ΔV2 envelope. (A) First, it was examined whether the captured V3 loop peptide interacts with specific anti-V3 loop MAbs recognizing linear (447D) and conformational (391-95D) V3 loop epitopes. (B) Next, the titer was determined of anti-V3 loop antibodies present in sera collected 2 and 4 weeks following the first and second boosts from the two vaccinated animals. As a comparison the titers of total anti-envelope antibodies present in the same sera were also included.

FIG. 13 A-B shows neutralization of HIV-1 of clades A, E and D by sera from two animals immunized with a HIV-1 clade B immunogen-derived modified envelope protein having a V2 region deletion.

FIG. 14 depicts the polynucleotide sequence of a full-length SF162ΔV2 gp140 envelope protein (SEQ ID No:1).

FIG. 15 depicts the polynucleotide sequence of a SF162ΔV2 gp140 envelope protein fragment (SEQ ID No:3).

FIG. 16 depicts the amino acid sequence of a full-length SF162ΔV2 gp140 envelope protein (SEQ ID No:2).

FIG. 17 depicts the amino acid sequence of a SF162ΔV2 gp140 envelope protein fragment (SEQ ID No:4).

### DETAILED DESCRIPTION OF THE INVENTION

The inventor herein has made the surprising discovery that animal immunization using modified HIV-1 envelope proteins having a deletion in the V2 (second hypervariable) region elicits potent neutralizing antibodies as part of an anti-HIV-1 envelope-specific immune response. Moreover, the immune response is directed not only to the wild-type form of the immunogen envelope protein, but to other HIV-1 viruses both within and outside of the clade from which the immunogen was derived. This potent, heterologous immune response and in particular the robust humoral response offers a new means for vaccination, among other immunotherapies, for the prophylaxis and treatment of HIV infection. The invention is directed to both DNA, viral and protein vaccines comprising one or more HIV-1 envelope proteins of fragments thereof having a deletion in the V2 region, and to methods for their use.

In one non-limiting embodiment, immunization may be carried out with DNA or virus encoding a HIV-1 envelope protein or fragment thereof having a deletion in the V2 region. As will be described in the examples below, a DNA vector capable of expressing a modified gp140 envelope protein from HIV-1 strain SF162 (clade B) was prepared which included a partial deletion in the V2 hypervariable region. In this instance, the first 27 N-terminal amino acids (81 nucleotides) of the DNA and protein sequence, respectively, were not expressed. These DNA and protein fragments of the modified gp140 of SF162 are provided in SEQ ID No:3 and SEQ ID No:4, respectively. The corresponding full-length sequences SEQ ID No:1 and SEQ ID No:2, respectively, are also useful for the same purposes. DNA immunization of macaques elicited immune responses including potent neutralizing antibodies. When depleted of CD8+ T lymphocytes and challenged with SHIV162P4, the vaccinated animals had lower peak viremias, exhibited rapid viral clearance from plasma, and showed delayed seroconversion, as compared to unimmunized, control animals. These results demonstrate the elicitation of a potent protective humoral response with the immunogen of the invention. Moreover, as mentioned above, cross-neutralizing reactivity against several heterologous HIV-1 strains was observed, supporting the utility of the V2 deletion immunogen in eliciting a general immune response against HIV-1 strains. In immunized rabbits, the modified (V2 deletion) immunogen was also more effective at eliciting neutralizing antibodies against the homologous, parental SF162 virus, but also against several heterologous HIV-1 isolates. In macaques, only the modified immunogen was capable of eliciting neutralizing antibodies against heterologous isolates.

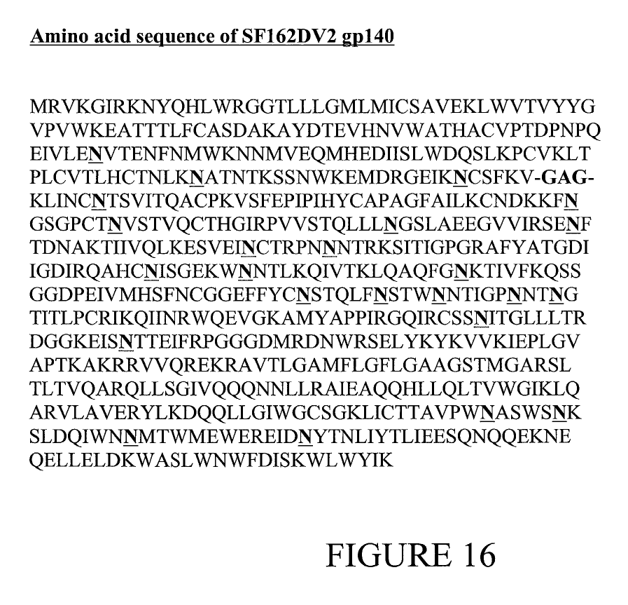

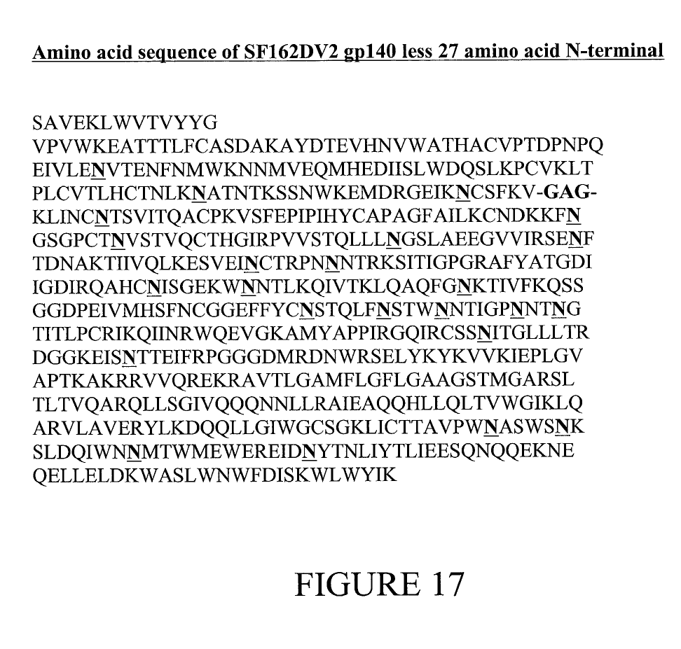

The present invention is directed to any type of or protocol for immunization, such as DNA, virus, protein, combinations thereof, and utilizing one or more adjuvants, or any combination of materials in addition to at least one of the immunogens described herein, and any immunization protocol employing as immunogen a protein or DNA encoding an HIV-1 viral envelope protein comprising a deletion in the V2 (second hypervariable) loop (also referred to herein interchangeably as the V2 domain or V2 region). The wild-type sequence of HIV-1 envelope protein candidates for a deletion in the V2 region in the protein, DNA or virus immunogen as described herein may be found at http://idiotype.lanl.gov/, and all such sequences are incorporated herein by reference in their entireties as starting sequences for the preparation of an immunogen. One or a combination of such immunogens may be used together. Furthermore, various further modifications of the modified (i.e., V2 loop deletion-containing) envelope proteins of the invention or DNA encoding the modified envelope proteins of the invention may be made without departing from the invention. For example, the DNA or viral nucleotide sequence encoding the native envelope leader peptide of the modified protein can be replaced with a signal peptide of, for example, the human tissue-specific plasminogen activator gene, for higher protein expression in the mammalian cells. Other signal peptides may be used. In another embodiment, a portion of the modified protein or its encoding DNA sequence may be truncated to provide an immunogen for producing a neutralizing humoral response, and such modifications are fully embraced herein. Preferably, a fragment is a truncation at the N-terminal end of the modified protein or DNA or virus encoding the modified protein, the truncation being from one up to about 30 amino acids, but it not so limiting, and other truncations are embraced which provide an immunogen with the immunological properties herein described. Moreover, expression of the DNA constructs in a mammalian cell, as shown in the examples herein, provides a glycosylated protein, glycosylated at the asparagine residues indicated in FIGS. 16 and 17, and the protein immunogen compositions embraced herein include the glycosylated forms of the protein. Thus, the foregoing non-limiting examples of variations in the protein and DNA immunogens of the invention which commonly comprise a deletion in the V2 loop domain are encompassed by the phrase modified protein or fragments thereof, or DNA or virus encoding the modified protein or fragments thereof.

The V2 domain is one of the five hypervariable regions of the gp120 subunit of the HIV envelope. Its length (number of amino acids) and extent of glycosylation vary among HIV isolates. In the case of the SF162 virus, the V2 loop comprises 40 amino acids. In the studies herein, 30 amino acids were eliminated from the central region of the V2 loop, replacing them by the GAG tripeptide. One of skill in the art may make other deletions in the V2 domain of this strain, or deletions in the V2 region in other strains, which exhibit the same immune-response-eliciting properties and may readily be evaluated for such properties, without deviating from the scope and spirit of the invention. As used herein, the abbreviation “ΔV2” refers to a partial or full deletion in the V2 domain. A detailed description of the V2 domain of HIV-1 may be found in Stamatatos, L., M. Wiskerchen, and C. Cheng-Mayer. 1998. Effect of major deletions in the V1 and V2 loops of a macrophage-tropic HIV-1 isolate on viral envelope structure, cell-entry and replication. AIDS Res. Hum. Retroviruses 14:1129-1139, which is incorporated herein by reference in its entirety.

One non-limiting means by which a modified protein or DNA encoding a modified protein comprising the HIV-1 envelope protein may be prepared with a deletion in the V2 region may be carried is that described in the aforementioned article or in Stamatatos, L., and C. Cheng-Mayer. 1998. An envelope modification that renders a primary, neutralization resistant, clade B HIV-1 isolate highly susceptible to neutralization by sera from other clades. J. Virol. 72:7840-7845. By way of non-limiting example, a modified V2 deletion of the envelope protein of HIV-1 SF162 (a clade B HIV-1) may be prepared, having the DNA and protein sequence depicted in SEQ ID No:1 and SEQ ID No:2, respectively. However, other clade B HIV-1 envelope proteins may be similarly modified and the protein or DNA encoding the protein used as immunogen. Alternatively, HIV-1 envelope proteins of other HIV-1 clades may be used. A selection of HIV-1 proteins and the amino acid sequences of their envelope proteins may be found in the literature, such as at the Los Alamos National Laboratories' HIV sequence database, accessible at http://idiotype.lanl.gov/. The present invention embraces these and other HIV-1 envelope proteins as candidates for deletions in the V2 region for the preparation of a DNA or protein immunogen for the purposes herein.

Standard molecular biological methods may be used to prepare the HIV-1 envelope protein with a deletion in the V2 domain, as well as the encoding DNA including viruses encoding the protein, and the invention herein is not limited as to the method by which the immunogen is prepared. As used herein, the term DNA vaccine includes and embraces a viral vaccine comprising DNA encoding the aforementioned protein. Such methods are well known in the art. As demonstrated herein, one of skill in the art can readily determine the ability of a DNA or protein immunogen of the invention to elicit a heterologous HIV-1 immune response in an animal. In the non-limiting example of the SF162 clade B HIV-1 viral strain, a 30-amino acid deletion from amino acids T160 to Y189 was prepared, the deleted sequence replaced with a Gly-Ala-Gly tripeptide. The replacement of the deleted sequences with the aforementioned tripeptide, or any short peptide, is not required, but may be done for expedience.

An animal in which the heterologous viral immune response may be raised is any animal susceptible to HIV-1 infection or a related virus. Such animals include but are not limited to humans, non-human primates, and other mammals. In the instance of humans, the methods of the invention may be carried out with HIV-1, HIV-2, etc.; in non-human primates, with SHIV-1.

The invention is also directed to a vaccine pharmaceutical composition is provided for immunizing an animal against HIV-1 virus, the vaccine pharmaceutical composition comprising an effective heterologous immune response-eliciting amount of at least one modified HIV-1 envelope protein or fragment thereof, DNA encoding the at least one modified HIV-1 envelope protein or fragment thereof, or a combination thereof, the modified envelope protein having a V2 region deletion; and a pharmaceutically-acceptable carrier or excipient. As used interchangeably herein, the immunogens may be the full-length or truncated forms of the modified protein or DNA encoding the modified protein, provided that the deletion in the V2 region elicits a heterologous immune response. Various selections of useful immunogens are described above. In one embodiment, the modified HIV-1 envelope protein or fragment is from a clade-B HIV-1 strain. In a preferred embodiment, the HIV-1 strain is SF162. By way of non-limiting examples, the modified HIV-1 envelope protein or fragment is SEQ ID No:2 or SEQ ID No:4; and a DNA encoding the at least one modified HIV-1 envelope protein or fragment is SEQ ID No:1 or SEQ ID No:3. Glycosylation of the protein or fragment as expressed in mammalian cells is also provided.

The vaccine pharmaceutical composition may comprise one or more of the foregoing DNA or protein immunogens, together with one or more pharmaceutically-acceptable carriers, excipients or diluent, to facilitate administration of the vaccine. Moreover, additional components, such as one or more adjuvants, may be included to enhance the immune response. The selection of the adjuvant will depend on the animal to be immunized, particularly in humans in which the selection of appropriate adjuvants is limited. One of skill in the art may select the appropriate pharmaceutically-acceptable components to include with the immunogen(s) to achieve the desired effect.

It is a further object of the present invention to provide a method for assessing whether a compound, such as an immunogen, is capable of generating protective antibodies against heterologous strains of HIV-1. The method is carried out by immunizing an animal with an immunogen, depleting the animal of its CD8+ T-lymphocytes, and then screening the animal for the presence at least of protective antibodies, and preferably the presence of protective antibodies, to at least one heterologous strain of HIV-1. The depleting may be carried out by administering to the animal anti-CD8 monoclonal antibodies. The compound may be an HIV-derived polypeptide or fragment thereof, such as but not limited to a DNA vaccine wherein the DNA vaccine encodes an HIV-derived polypeptide or fragment thereof. The immunization protocol may comprise a DNA vaccine, a viral vaccine, a protein, any fragments thereof, any combination thereof, and a protocol in which either or both are administered sequentially in order to induce an immune response. In a non-limiting embodiment, the neutralizing antibodies are protective antibodies. The method in which eliciting of protective antibodies is evaluated may be carried out in an animal such as a primate or other animal capable of generating protective antibodies to HIV, but it is not so limiting. As noted above, the foregoing method may be utilized to assess the effectiveness of a DNA and/or protein immunogen of the invention.

As described in the examples below, the observation that the lowest levels of peak plasma viremia were recorded in a animal vaccinated with the ΔV2 immunogen and whose serum had the strongest neutralizing activity against SHIV162P4 at the day of challenge, indicates that neutralizing antibodies played an important protective role during the first 7 days post-challenge. The fact that strong anamnestic anti-HIV envelope responses were developed immediately following SHIV62P4-challenge indicates that antibodies contributed to the rapid viral-clearance to undetectable levels. However, because the CD8+ lymphocytes reappeared in the periphery of the vaccinated animals 7 days post-challenge, they may also have contributed to this rapid viral clearance.

Moreover, the herein studies also show an immune response to HIV-1 of different clades than that from which the immunogen was prepared, referred to herein as a heterologous immune response.

These studies highlight the important protective role of non-CD8-mediated DNA-based vaccine-induced anti-HIV envelope responses and demonstrate the feasibility to develop an effective anti-HIV vaccine for human use for the prophylaxis and treatment of HIV infection. As noted above, the strategy of using a modified envelope protein with a ΔV2 loop deletion is a strategy that may be employed for any V2-loop-bearing envelope protein, and the present invention embraces any and all such uses, as well as pharmaceutical compositions comprising a ΔV2 loop deletion modified protein or DNA vaccine, or combination, for the purposes of eliciting an immune response.

In the studies described herein, immunogenicity was compared between soluble oligomeric gp140 envelope proteins derived from related neutralization-resistant (SF162) and neutralization-susceptible (SF162ΔV2) viruses (Stamatatos, L., and C. Cheng-Mayer. 1998. An envelope modification that renders a primary, neutralization resistant, clade B HIV-1 isolate highly susceptible to neutralization by sera from other clades. J. Virol. 72:7840-7845). The only difference between the two immunogens is the absence of 30 amino acids from the V2 loop of the SF162ΔV2-derived immunogen (Stamatatos, L., and C. Cheng-Mayer. 1998. An envelope modification that renders a primary, neutralization resistant, clade B HIV-1 isolate highly susceptible to neutralization by sera from other clades. J. Virol. 72:7840-7845). Immunization studies were first performed in rabbits, where it was observed that although both proteins elicited similar titers of binding antibodies, the modified immunogen elicited higher titers of neutralizing antibodies against isolates expressing not only the modified SF162ΔV2 envelope, but also the unmodified parental SF162 envelope.

In rabbits, both the unmodified SF162gp140 and the modified ΔV2gp140 immunogens elicited neutralizing antibodies against several heterologous primary HIV-1 isolates, but the potential of the modified immunogen to do so was greater, and importantly, not previously described or expected. Thus, not only a greater number of animals vaccinated with the modified immunogen elicited cross-reactive neutralizing antibodies, but also the breadth and potency of the cross-neutralizing responses were higher in sera collected from these animals than animals immunized with the unmodified immunogen. The modified immunogen more effectively elicits antibodies recognizing neutralization epitopes that are conserved among several HIV isolates than the unmodified immunogen.

The vaccination studies conducted in rhesus macaques confirm the observations made in rabbits, that the modified ΔV2gp140 immunogen is more effective than the unmodified SF162gp140 in eliciting neutralizing antibodies against isolates expressing the parental SF162 envelope. Importantly, in macaques only the modified envelope was capable of eliciting neutralizing antibodies against heterologous HIV-1 isolates.

The present invention embraces other envelope modifications in addition to the ΔV2 loop deletion described herein. Such modifications are expected to increase the exposure and/or the number of conserved neutralization epitopes on the immunogen.

The following examples are presented in order to more fully illustrate the preferred embodiments of the invention. They should in no way be construed, however, as limiting the broad scope of the invention.

### EXAMPLE 1

Two Rhesus macaques (Rh) (H445 and J408) were immunized both intradermally and intramuscularly at weeks 0, 4 and 8 with a DNA vector (Chapman, B. S., R. M. Thayer, K. A. Vincent, and N. L. Haigwood. 1991. Effect of intron A from human cytomegalovirus (Towne) immediate-early gene on heterologous expression in mammalian cells. Nucleic Acids Res. 19:3979-86; zur Megede, J., M. C. Chen, B. Doe, M. Schaefer, C. E. Greer, M. Selby, G. R. Otten, and S. W. Barnett. 2000. Increased expression and immunogenicity of sequence-modified human immunodeficiency virus type 1 gag gene. J Virol. 74:2628-35) (2 mg total DNA each time) expressing the SF162ΔV2 gp140 envelope with an intact gp120-gp41 cleavage site (Stamatatos, L., M. Lim, and C. Cheng-Mayer. 2000. Generation and structural analysis of soluble oligomeric envelope proteins derived from neutralization-resistant and neutralization-susceptible primary HIV-1 isolates. AIDS Res. and Human Retroviruses. 16:981-994). The DNA construct was codon-optimized for high expression in mammalian cells. At week 27 the animals were immunized one additional time with DNA and with the CHO-produced, purified oligomeric SF162ΔV2 gp140 protein (100 μg) mixed with the MF-59C adjuvant. At week 38 the animals were immunized one additional time with the adjuvanted protein alone.

The development of binding antibodies was evaluated by ELISA methodologies (Stamatatos, L., and C. Cheng-Mayer. 1995. Structural modulations of the envelope gp120 glycoprotein of human immunodeficiency virus type 1 upon oligomerization and differential V3 loop epitope exposure of isolates displaying distinct tropism upon virion-soluble receptor binding. J. Virol. 69:6191-6198).

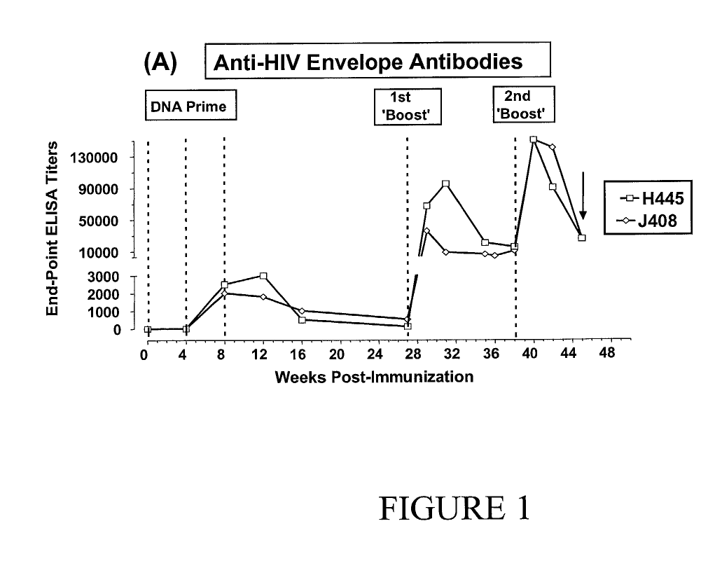

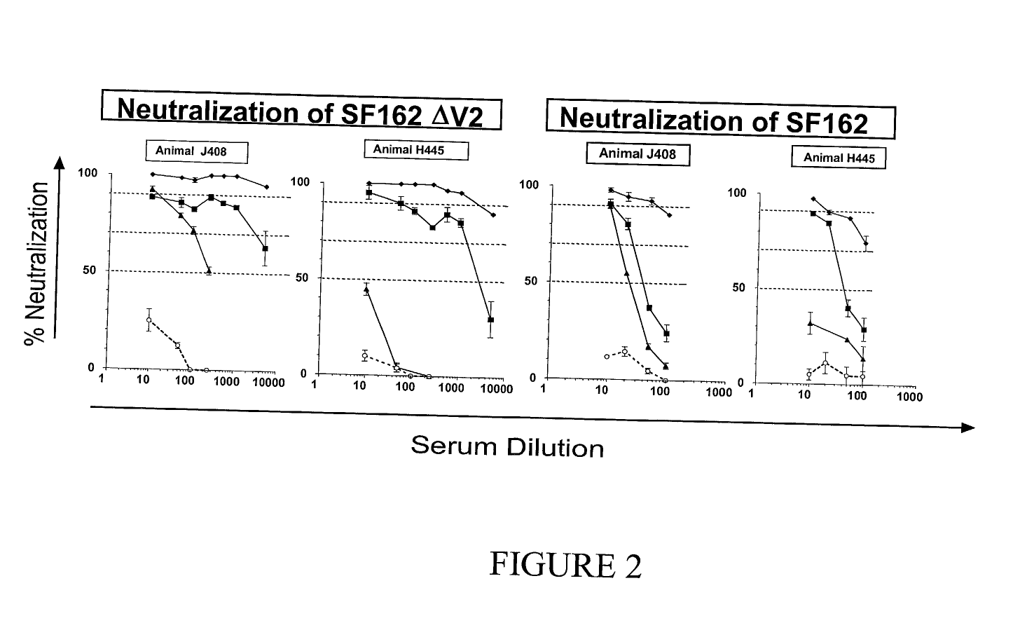

Antibodies were detectable following the second DNA immunization and their titers did not increase following the third DNA immunization (FIG. 1). During the following five months the titers decreased gradually, but were always detectable. The first ‘boost’ increased the titers by approximately 1-2 log₁₀ from the peak value recorded following the third DNA immunization. The titers gradually decreased and leveled off during the following 11 weeks, at which point the animals received a second ‘boost’, which further increased the antibody titers. Neutralizing antibodies (NA) were evaluated using the ‘activated PBMC-target’ assay (Stamatatos, L., and C. Cheng-Mayer. 1998. An envelope modification that renders a primary, neutralization resistant, clade B HIV-1 isolate highly susceptible to neutralization by sera from other clades. J. Virol. 72:7840-7845), using pre-immunization sera to correct for non-specific neutralization (FIG. 2). Following the third DNA-immunization, the NA titers in animal H445 were lower than those in animal J408, even though the binding antibody titers were similar between the two animals. The NA titers against both SF 1 62ΔV2 and SF162 increased significantly during the subsequent ‘boosts’. Vaccine-specific proliferative responses were also recorded in both animals. Stimulation indexes (S.I.) of 5 and 10 were recorded following the first ‘boost’ in animals J408 and H445, respectively. The second ‘boost’ increased the potency of these responses in animal H445 (S.I. of 25), but not in animal J408 (S.I. of 5).

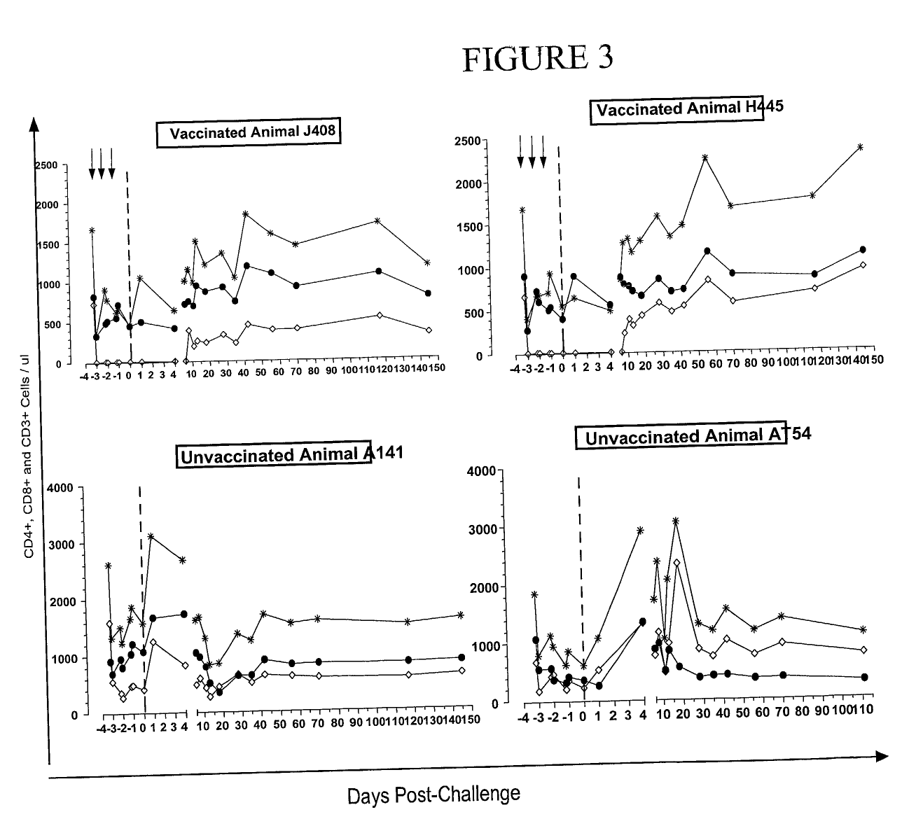

To evaluate the protective role of the anti-HIV envelope antibodies elicited by the vaccine of the invention, CD8+ cells were depleted from the vaccinated animals prior to viral-challenge (FIG. 3). CD8-depletion was achieved by three intravenous administrations of the anti-CD8 MAb OKT8F (2 mg/kg) at daily intervals (Jin, X., D. E. Bauer, S. E. Tuttleton, S. Lewin, A. Gettie, J. Blanchard, C. E. Irwin, J. T. Safrit, J. Mittler, L. Weinberger, L. G. Kostrikis, L. Zhang, A. S. Perelson, and D. D. Ho. 1999. Dramatic rise in plasma viremia after CD8(+) T cell depletion in simian immunodeficiency virus-infected macaques. J Exp Med. 189:991-8). CD8+ T lymphocytes remained undetectable for approximately 10 days. Concomitantly, a decrease was recorded in the total number of circulating CD3+ T cells. This indicates that the recorded depletion of CD8+ T cells from the periphery is due to their actual elimination. Although CD8-depletion from the lymph nodes was not evaluated, it was previously demonstrated that a concomitant depletion of CD8+ T cells from the periphery and lymph nodes occurs when anti-CD8 MAbs are introduced in the blood circulation of macaques (Matano, T., R. Shibata, C. Siemon, M. Connors, H. C. Lane, and M. A. Martin. 1998. Administration of an anti-CD8 monoclonal antibody interferes with the clearance of chimeric simian/human immunodeficiency virus during primary infections of rhesus macaques. J. Virol. 72:164-169; Schmitz, J. E., M. J. Kuroda, S. Santra, V. G. Sasseville, M. A. Simon, M. A. Lifton, P. Racz, K. Tenner-Racz, M. Dalesandro, B. J. Scallon, J. Ghrayeb, M. A. Forman, D. C. Montefiori, E. P. Rieber, N. L. Letvin, and K. A. Reimann. 1999. Control of viremia in simian immunodeficiency virus infection by CD8+ lymphocytes. Science. 283:857-60).

One day following the last administration of OKT8F, the immunized and two un-immunized naive animals were challenged intravenously with 100 TCID₅₀ of a cell-free stock of the SHIV162P4 virus (Harouse, J. M., A. Gettie, R. C. Tan, J. Blanchard, and C. Cheng-Mayer. 1999. Distinct pathogenic sequela in rhesus macaques infected with CCR5 or CXCR4 utilizing SHIVs. Science. 284:816-9). This isolate was neutralized by 50% and 90% by sera (1:5 dilution) collected at the day of challenge from animals H445 and J408, respectively.

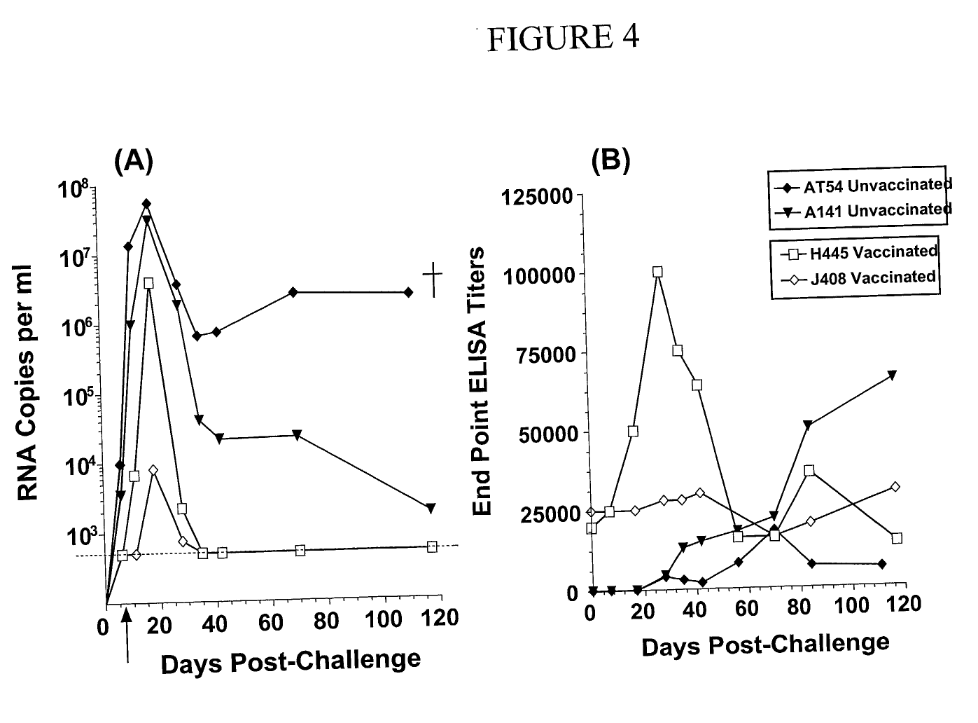

Both vaccinated and unvaccinated animals became infected; however, differences in the peak viral load levels and viral set points were noted between the two groups (FIG. 4A). Eleven days post-challenge, plasma viremia in the vaccinated animal H445 was lower by 2 and 4 log₁₀ as compared to that of the unvaccinated animals A141 and AT54, respectively, while the vaccinated animal J408 was aviremic. At peak viremia, viral plasma levels in the vaccinated animals were 1-4 log₁₀ lower than in the unvaccinated animals. Following peak viremia, an initial rapid decrease followed by a more gradual decrease in plasma viral loads was recorded in the unvaccinated animal A141, while sustained high viral loads were recorded in the second unvaccinated animal AT54. A very rapid decrease to undetectable levels was recorded in both vaccinated animals within 35 days post-challenge.

Concomitant with the appearance of plasma viremia in the vaccinated animal H445, a rapid increase (by approximately 5 fold) of the anti-HIV envelope antibody titers was monitored (FIG. 4B). Subsequently, as the viral load in this animal decreased to undetectable levels, the antibody titers gradually decreased to pre-challenge titers. In contrast, the anti-envelope antibody titers did not increase in the second vaccinated animal J408, which had the lowest levels of peak plasma viremia. In the unvaccinated animals, anti-HIV envelope antibodies became detectable approximately 30 days post-challenge. Although their titers increased over time in animal A141 they remained weak and eventually declined prior to death in animal AT54.

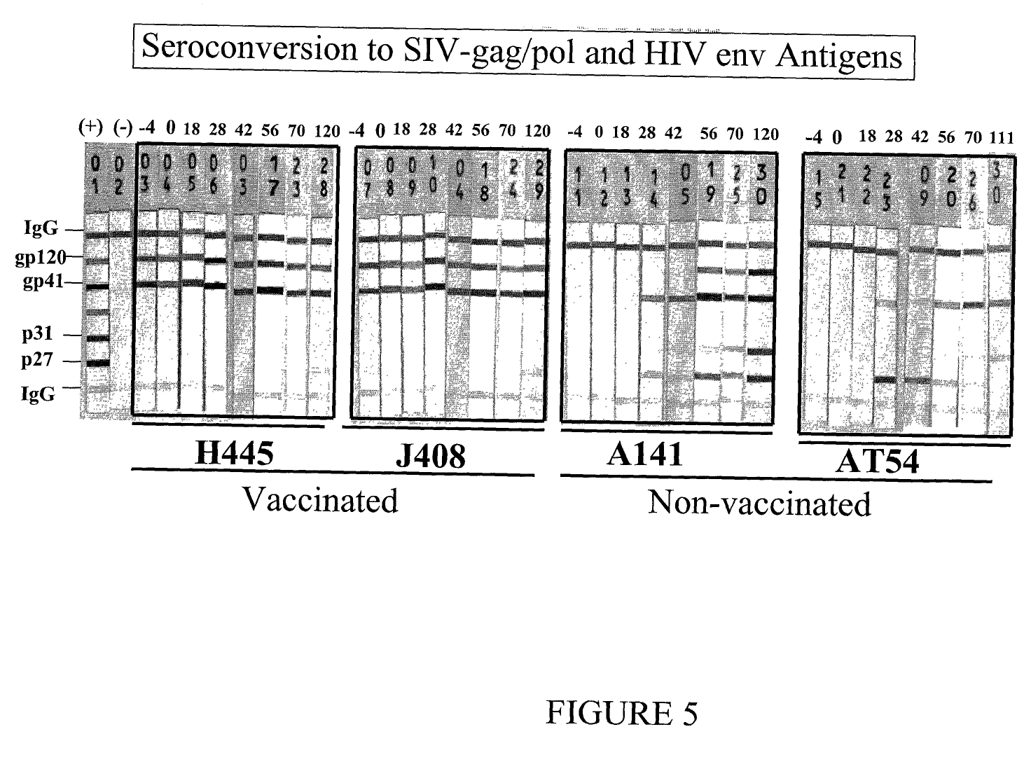

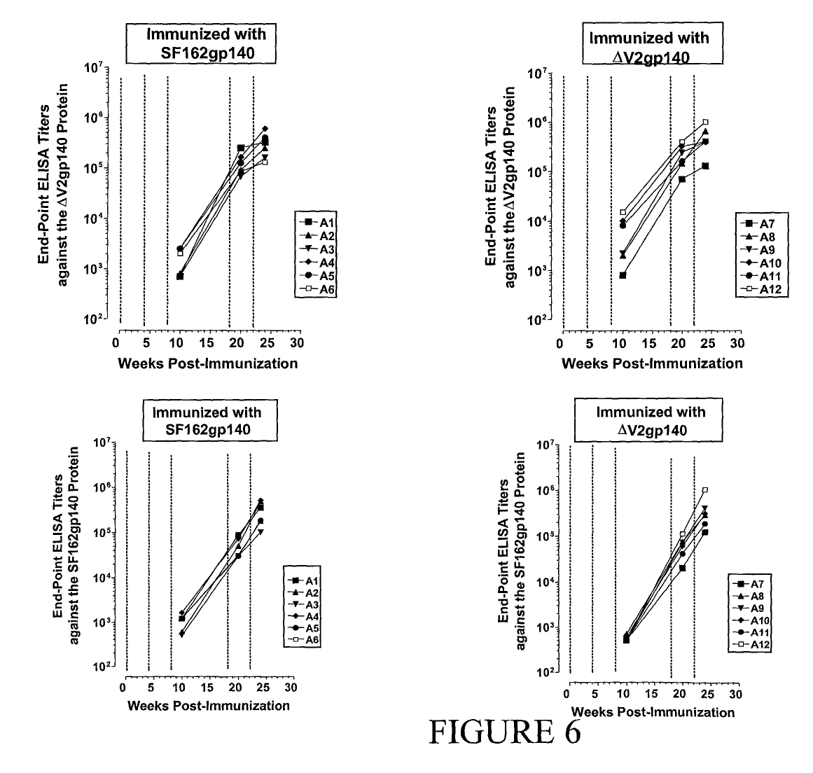

The two unvaccinated animals seroconverted to SIV gag p27 and pol 31 proteins within 2 weeks post-challenge, while the two vaccinated animals remained seronegative for the first 17 weeks post-challenge (FIG. 5). This figures shows seroconversion to the core SIV proteins gag p27 and pol p31, as well as to the gp41 and gp120 HIV envelope subunits, and was determined with RIBATM. The numbers above each strip indicate the days at which serum samples were collected relative to the day of challenge (day 0) [(+) positive control strip; (−) negative control strip].

Also, although virus was recoverable from Rh-PBMC collected from the unvaccinated animals at 18, 42 and 48 days post-challenge, it was only recoverable at day 18 from the vaccinated animals. Finally, in contrast to the two vaccinated animals and the unvaccinated animal A141, which remained healthy, the second unvaccinated animal AT54 died from SAIDS 16 weeks post-challenge.

The observation that the lowest levels of peak plasma viremia were recorded in the vaccinated animal J408 whose serum had the strongest neutralizing activity against SHIV162P4 at the day of challenge, suggests that neutralizing antibodies played an important protective role during the first 7 days post-challenge. However, in addition to neutralizing antibodies, envelope-specific antibodies without neutralizing activity may have been elicited by the vaccine of the invention and may also have contributed in viral clearance. The fact that strong anamnestic anti-HIV envelope responses were developed in animal H445 immediately following SHIV-challenge indicates that antibodies contributed to the rapid viral-clearance to undetectable levels. However, because the CD8+ lymphocytes reappeared in the periphery of the vaccinated animals 7 days post-challenge, they may also have contributed to this rapid viral clearance.

These studies highlight the important protective role of non-CD8-mediated DNA-vaccine-induced anti-HIV envelope responses and demonstrate the feasibility to develop an effective anti-HIV vaccine.

### EXAMPLE 2

In the studies presented here, the immunogenic potential of the unmodified SF162 is compared to that of modified SF162ΔV2 (from here on designated as ΔV2) envelopes. Using the gene-gun vaccination methodology rabbits were immunized with the gp140 form of the SF162 and ΔV2 envelopes. Both immunogens elicited the generation of similar antibody titers, but the modified immunogen elicited higher titers of neutralizing antibodies against the parental SF162 virus than the unmodified immunogen. Additionally, the ΔV2-derived modified immunogen was more effective than the SF162-derived unmodified immunogen in generating antibodies capable of neutralizing heterologous primary HIV-1 isolates.

The immunogenicity of these two antigens was also evaluated in Rhesus macaques, an animal model more closely related to humans and more suitable for HIV-vaccine studies, using the DNA-prime followed by protein-boosting vaccination methodology. Here too the modified immunogen was found to be more effective than the unmodified immunogen in generating potent neutralizing antibodies both against the homologous SF162ΔV2 and parental SF162 viruses. The antibodies elicited in macaques by the modified, but not unmodified, immunogen neutralized several heterologous primary HIV-1 isolates. These studies indicate for the first time that potent cross-reactive neutralizing antibodies can be elicited in non-human primates immunized with soluble oligomeric subunit HIV envelope vaccines derived from an R5-using primary-like HIV-1 isolate. They support the use of specific envelope modifications to increase the exposure of neutralization epitopes and increase the breadth and potency of these responses.

Viruses: The isolation and phenotypic characterization of the SF162 and SF162V2 isolates was previously reported (Cheng-Mayer, C., M. Quiroga, J. W. Tung, D. Dina, and J. A. Levy. 1990. Viral determinants of human immunodeficiency virus type 1 T-cell or macrophage tropism, cytopathogenicity, and CD4 antigen modulation. J. Virol. 64:4390-4398; Stamatatos, L., and C. Cheng-Mayer. 1998. An envelope modification that renders a primary, neutralization resistant, clade B HIV-1 isolate highly susceptible to neutralization by sera from other clades. J. Virol. 72:7840-7845). The primary clade B HIV-1 isolates 92US660, 92HT593, 92US657, 92US714, 92US727, 91US056, 91US054 and 93US073 were obtained from the NIH AIDS Research and Reference Reagent Program. All viral stocks were prepared and titrated in activated human peripheral blood mononuclear cells (PBMC).

Vaccines: The DNA vector used to express the immunogens of the invention in rabbits is the pJW4303 (Lu, S., R. Wyatt, J. F. L. Richmond, F. Mustafa, S. Wang, J. Weng, D. C. Montefiori, J. Sodroski, and H. L. Robinson. 1998. Immunogenicity of DNA vaccines expressing human immunodeficiency virus type 1 envelope glycoprotein with and without deletions in the V1/V2 and V3 regions. AIDS Res. Hum. Retroviruses 14:151-155). The DNA vector used to immunize Rhesus macaques is derived from the pCMVKm2 vector (Chapman, B. S., R. M. Thayer, K. A. Vincent, and N. L. Haigwood. 1991. Effect of intron A from human cytomegalovirus (Towne) immediate-early gene on heterologous expression in mammalian cells. Nucleic Acids Res. 19:3979-86; zur Megede, J., M. C. Chen, B. Doe, M. Schaefer, C. E. Greer, M. Selby, G. R. Otten, and S. W. Barnett. 2000. Increased expression and immunogenicity of sequence-modified human immunodeficiency virus type 1 gag gene. J. Virol. 74:2628-35). Both DNA plasmids contain the human CMV enhancer/promoter elements and the native leader peptide of the HIV envelope was replaced with that derived from the tissue-specific plasminogen activator gene. In the case of macaque-immunizations, the DNA construct was codon-optimized for high expression in mammalian cells. Both DNA vectors express the gp140 ectodomain form of the HIV envelope immunogen, with an intact gp120-gp41 cleavage site.

Protein-boosting immunizations were performed only in rhesus macaques to increase the titer of antibodies elicited following the DNA-phase of immunization. For this purpose, the ΔV2 gp140 protein was produced in CHO cells and purified as stable soluble trimers. To increase, however, the stability of these secreted oligomers, the gp120-gp41 cleavage site was eliminated by mutagenesis (Earl, P. L., S. Koenig, and B. Moss. 1991. Biological and immunological properties of human immunodeficiency virus type 1 envelope glycoprotein: analysis of proteins with truncations and deletions expressed by recombinant vaccinia viruses. J Virol 65:31-41; Earl, P. L., and B. Moss. 1993. Mutational analysis of the assembly domain of the HIV-1 envelope glycoprotein. AIDS Res. Hum. Retroviruses 9:589-594; Stamatatos, L., M. Lim, and C. Cheng-Mayer. 2000. Generation and structural analysis of soluble oligomeric envelope proteins derived from neutralization-resistant and neutralization-susceptible primary HIV-1 isolates. AIDS Res. Hum. Retroviruses 16:981-994).

Immunizations: a) Rabbits: Using the gene-gun vaccination methodology (Lu, S., R. Wyatt, J. F. L. Richmond, F. Mustafa, S. Wang, J. Weng, D. C. Montefiori, J. Sodroski, and H. L. Robinson. 1998. Immunogenicity of DNA vaccines expressing human immunodeficiency virus type 1 envelope glycoprotein with and without deletions in the V1/V2 and V3 regions. AIDS Res. Hum. Retroviruses 14:151-155) the animals received 5 DNA immunizations (each immunization consisting of 36 shots of 0.5 μg DNA each) at weeks 0, 4, 8, 18 and 22. Blood was drawn two weeks following each immunization. Six animals (A1-A6) were immunized with the unmodified SF162gp140 immunogen and six animals (A7-A12) with the modified ΔV2gp140 immunogen. Two animals (A13 and A14) served as controls and were immunized with the DNA vector alone.

b) Rhesus macaques: Animals H445 and J408 were immunized with the modified ΔV2gp140 immunogen, animals N472 and P655 with the unmodified SF162gp140 immunogen and animals M844 and H473 with the DNA vector alone. Prior to the initiation of immunizations, the animals were tested for antibodies to various Simian viruses such as SIV, type D retroviruses and STLV-1. Animals vaccinated with the modified envelope were immunized with DNA at weeks 0, 4 and 8, and animals vaccinated with the unmodified envelope were immunized with DNA at weeks 0, 4 and 9. The DNA (2 mg DNA in 1 ml of endotoxin-free water each time per animal) was administered both intradermally (i.d.) at two sites (2×0.2 mg) and intramuscularly (i.m.) (2×0.8 mg in the quadriceps muscles). Animals were immunized a fourth time with DNA and at the same time with the purified oligomeric ΔV2gp140 or SF162gp140 proteins mixed with the MF-59C adjuvant. The proteins (0.1 mg of purified protein in 0.5 ml total volume per animal) were administered i.m. in the deltoids. The control animals received only adjuvant. This DNA plus protein ‘booster’ immunization took place at week 27 for animals vaccinated with the modified immunogen and at week 48 for animals immunized with the unmodified immunogen. At week 38 the animals immunized with the modified, but not those immunized with the unmodified, immunogen were immunized one additional time with the adjuvanted protein alone (no DNA).

Antibody determination: a) Anti-gp140 antibodies: Titers were determined throughout the immunization protocol using an ELISA methodology as previously described (Stamatatos, L., and C. Cheng-Mayer. 1995. Structural modulations of the envelope gp120 glycoprotein of human immunodeficiency virus type 1 upon oligomerization and differential V3 loop epitope exposure of isolates displaying distinct tropism upon virion-soluble receptor binding. J. Virol. 69:6191-6198; Stamatatos, L., M. Wiskerchen, and C. Cheng-Mayer. 1998. Effect of major deletions in the Vl and V2 loops of a macrophage-tropic HIV-1 isolate on viral envelope structure, cell-entry and replication. AIDS Res. Hum. Retroviruses 14:1129-1139). Briefly, purified soluble oligomeric ΔV2gp140 and SF162gp140 proteins were used to coat ELISA plates (Immulon 2HB) (0.2 μg of protein in 0.1 ml of 100 mM NaHCO3, pH 8.5) by an overnight incubation at 4° C. Non-adsorbed protein molecules were removed by washing with TBS and the wells were blocked with SuperBlock (SB) (Pierce). Heat-inactivated (56° C for 35 minutes) sera collected from the immunized animals were serially diluted in SB and added to the wells (0.1 ml per well) for one hour at 37° C. In the case of rabbits, sera from control animals receiving the DNA vector alone were used as negative controls. In the case of macaques, pre-immunization sera were used as negative controls. Unbound antibodies were removed by TBS-washing and the envelope-bound antibodies were detected with the use of goat anti-human (in the case of Rhesus sera) or anti-rabbit (in the case of rabbit sera) IgG coupled to alkaline phosphatase antibodies (Zymed Immunochemicals) as previously described (Stamatatos, L., and C. Cheng-Mayer. 1995. Structural modulations of the envelope gp120 glycoprotein of human immunodeficiency virus type 1 upon oligomerization and differential V3 loop epitope exposure of isolates displaying distinct tropism upon virion-soluble receptor binding. J. Virol. 69:6191-6198). The OD490 nm of each well was recorded with a Bioluminometer (Molecular Dynamics). A plot of the OD490 nm signals versus serum-dilution was generated and end-point antibody titers were determined as the highest post-immunization serum dilution that produces an OD490 nm value three times that of the OD 490 nm produced by the pre-immunization sera at their lowest dilution. Sera from various stages of immunization were tested at the same time.

Neutralization assays: Neutralization assays were performed using as target cells human PBMC activated for three days with PHA (Sigma, 3 μg/ml) as previously described (Mascola, J. R., M. G. Lewis, G. Stiegler, D. Harris, T. C. VanCott, D. Hayes, M. K. Louder, C. R. Brown, C. V. Sapan, S. S. Frankel, Y. Lu, M. L. Robb, H. Katinger, and D. L. Birx. 1999. Protection of Macaques against pathogenic simian/human immunodeficiency virus 89.6PD by passive transfer of neutralizing antibodies. J. Virol. 73:4009-18; Mascola, J. R., S. W. Snyder, O. S. Weislow, S. M. Belay, R. B. Belshe, D. H. Schwartz, M. L. Clements, R. Dolin, B. S. Graham, G. J. Gorse, M. C. Keefer, M. J. McElrath, M. C. Walker, K. F. Wagner, J. G. McNeil, F. E. McCutchan, D. S. Burke and the NIAID AIDS vaccine evaluation group. 1996. Immunization with envelope subunit vaccine products elicits neutralizing antibodies against laboratory-adapted but not primary isolates of human immunodeficiency virus type 1. J. Infect. Dis. 173:340-348; Stamatatos, L., and C. Cheng-Mayer. 1998. An envelope modification that renders a primary, neutralization resistant, clade B HIV-1 isolate highly susceptible to neutralization by sera from other clades. J. Virol. 72:7840-7845; Stamatatos, L., S. Zolla-Pazner, M. Gorny, and C. Cheng-Mayer. 1997. Binding of antibodies to virion-associated gp120 molecules of primary-like human immunodeficiency virus type 1 (HIV-1) isolates: effect on HIV-1 infection of macrophages and peripheral blood mononuclear cells. Virology 229:360-369). All HIV-1 isolates tested were grown and titrated in human PBMCs, aliquoted and kept frozen at −80° C. until further use. Viruses (50-100 TCID₅₀ in 50 μl of complete RPMI media containing 20 U/ml of IL-2 (Hoffmann-La Roche)) were pre-incubated with an equal volume of serially diluted heat-inactivated (35 minutes at 56° C.) sera for one hour at 37° C., in 96 well U-bottom plates (Corning). For each serum dilution, triplicate wells were used. Pre-immunization sera from macaques and sera collected from rabbits immunized with the DNA vector alone were also incubated with the viruses and served as controls for non-specific neutralization. To each well, 0.1 ml of complete media containing 0.4×10⁶ PHA-activated PBMC was added. Following an overnight incubation at 37° C., half the volume of each well was replaced with fresh, complete RPMI media. Following centrifugation of the plates (5 minutes at 2,000 rpm), half the volume of each well was again replaced with fresh media. This procedure was repeated twice. The p24 antigen concentration in each well was evaluated at various points following infection (usually at days 4, 6 and 11), using an in-house ELISA p24-detection assay. The mean percent neutralization from triplicate wells and the standard deviation for each serum dilution were calculated based on p24 concentrations recorded in wells containing virus, cells and no rabbit or macaque serum, as previously described (Stamatatos, L., S. Zolla-Pazner, M. Gomy, and C. Cheng-Mayer. 1997. Binding of antibodies to virion-associated gp120 molecules of primary-like human immunodeficiency virus type 1 (HIV-1) isolates: effect on HIV-1 infection of macrophages and peripheral blood mononuclear cells. Virology 229:360-369). However, it was noticed that infection of some isolates was reduced in the presence of pre-immunization sera (non-specific neutralization). The results are therefore presented from the neutralization studies in two ways. One, in the same figure both the neutralization curve recorded with sera collected prior to vaccination (pre-bleeds) is presented, and that recorded with sera collected at various stages following vaccination. Two, for each serum dilution the difference was calculated between the percent neutralization recorded with post-vaccination sera minus that recorded with pre-vaccination sera. In some figures, this difference (which is termed here “specific neutralization”) is plotted as a function of serum dilution. In parallel, the susceptibility was evaluated of the various primary isolates to neutralization by MAbs 2F5 and 2G12.

During these neutralization experiments the ability of sera collected from macaques immunized with the recombinant SF2 gp120 envelope was also evaluated. This immunogen was previously tested as a potential vaccine against HIV and failed to raise cross-reactive neutralizing antibodies (Mascola, J. R., S. W. Snyder, O. S. Weislow, S. M. Belay, R. B. Belshe, D. H. Schwartz, M. L. Clements, R. Dolin, B. S. Graham, G. J. Gorse, M. C. Keefer, M. J. McElrath, M. C. Walker, K. F. Wagner, J. G. McNeil, F. E. McCutchan, D. S. Burke and the NIAID AIDS vaccine evaluation group. 1996. Immunization with envelope subunit vaccine products elicits neutralizing antibodies against laboratory-adapted but not primary isolates of human immunodeficiency virus type 1. J. Infect. Dis. 173:340-348).

Results: Generation of antibodies in rabbits: Both the SF162- and ΔV2-derived immunogens elicited high titers of antibodies capable of binding to both the oligomeric ΔV2gp140 and SF162gp140 proteins (FIG. 6). As expected, variations in the antibody-titers were recorded throughout the vaccination schedule in animals belonging to either group. However, no statistically significant differences in antibody titers were recorded between the two animal groups throughout the immunization schedule. The antibody titers in each animal, regardless of whether it was immunized with the modified or the unmodified immunogen, were very weak during the first two immunizations (at 0 and 4 weeks). The fourth immunization (at 18 weeks) resulted in an increase in antibody titers, as compared to the third immunization (8 weeks), between 2 and 3 log10 in both animal groups. The fifth immunization (22 weeks) increased the antibody titers, as compared to the fourth immunization, against the SF162gp140 antigen (by less than 1 log₁₀), but not against the ΔV2gp140 protein. At the end of the vaccination schedule, very potent end-point ELISA binding antibody titers in the order of 10⁵-10⁶ were recorded in both animal groups against both antigens. Thus, it appears that in rabbits, based on the assay used here to determine antibody titers, the modified immunogen is as effective as the unmodified immunogen in eliciting the generation of antibodies even though the former immunogen lacks 30 amino acids from the V2 loop.

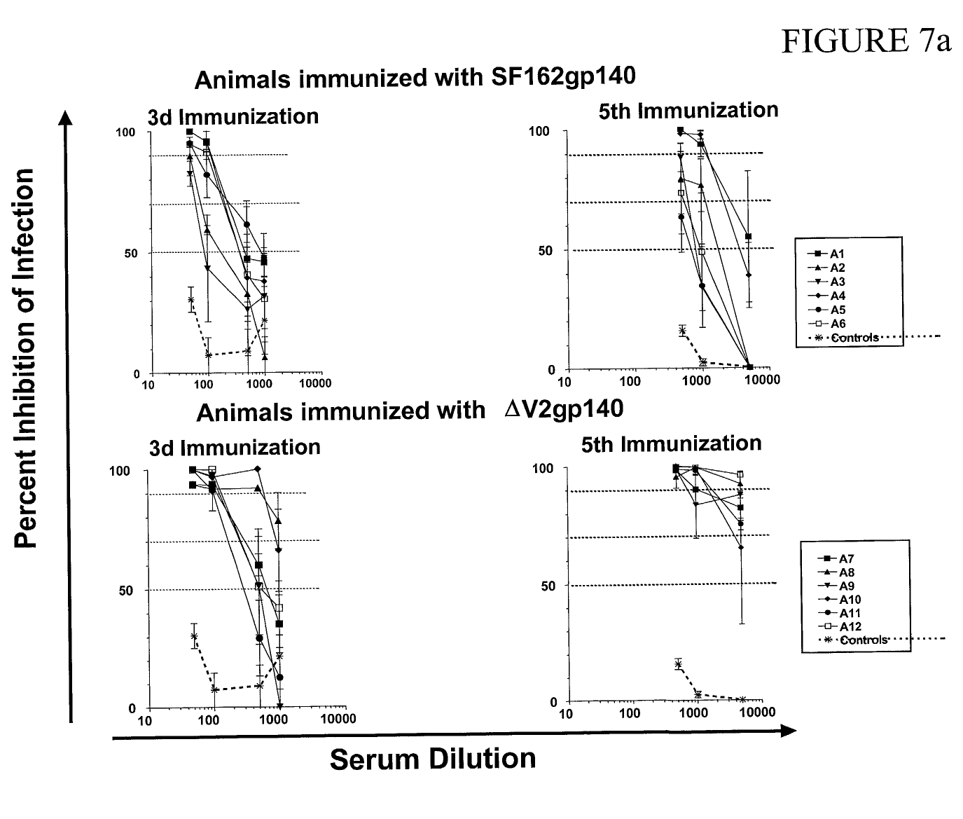

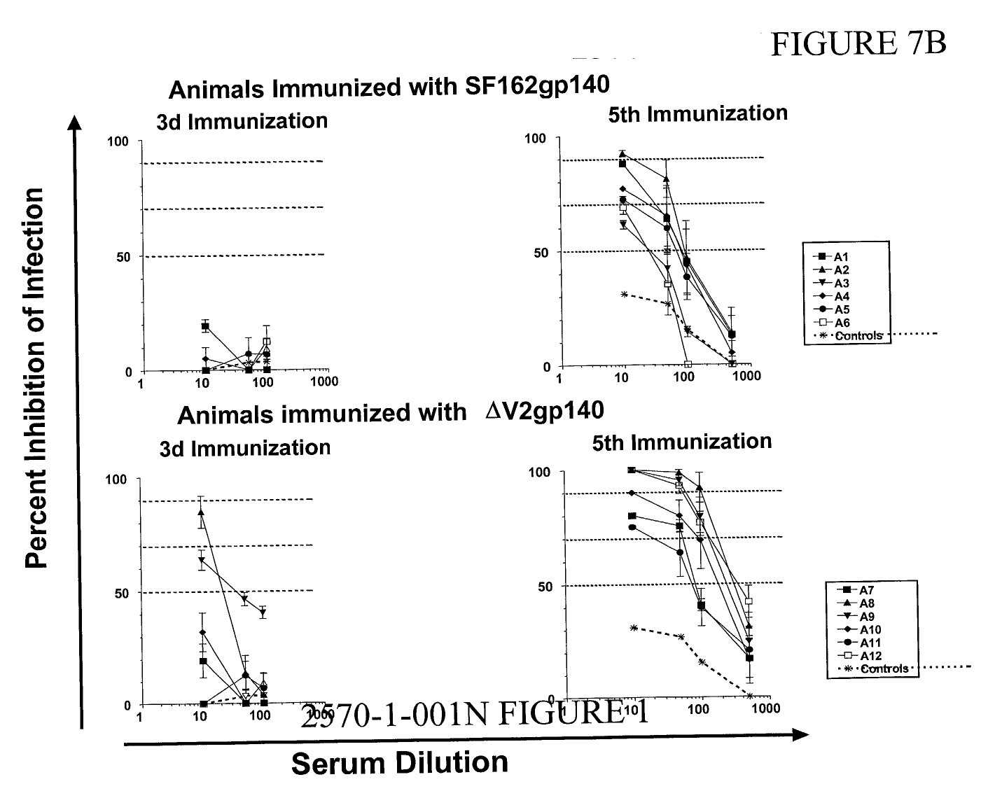

Neutralizing activity in rabbit sera against the SF162 and SF162ΔV2 isolates: Both immunogens generated neutralizing antibodies against the SF162ΔV2 virus following the third DNA-immunization (FIG. 7A). A trend towards higher neutralization titers in the modified immunogen-vaccinated group was recorded. Thus, the mean serum dilution at which 70% inhibition of infection was recorded (and standard error) for SF162gp140- and ΔV2gp140-immunized animals was 179 (+/−34) and 483 (+/−148), respectively. At this stage of vaccination, while 2 (A8 and A9) out of 6 animals immunized with the modified immunogen elicited neutralizing antibodies against the parental SF162 isolate, none of the animals immunized with the unmodified immunogen elicited antibodies capable of doing so (FIG. 7B). However, the number of animals that generated neutralizing antibodies against the SF162 and SF162ΔV2 viruses increased with each subsequent immunization, so that at the end of the immunization schedule (i.e., after the fifth immunization) all animals had generated neutralizing antibodies against the SF162 virus. In addition, the neutralization potency of each serum, regardless of whether the animal was vaccinated with the modified or unmodified immunogen, increased with each immunization.

At the end of the immunization schedule, sera collected from rabbits immunized with the modified immunogen had higher neutralization potency against the SF162ΔV2 as well as against SF162 viruses, than the sera collected from animals immunized with the unmodified immunogen. Six out of six animals immunized with the modified immunogen elicited antibodies capable of neutralizing the SF162ΔV2 virus between 70% and 100% at a 1:5,000 dilution (FIG. 7A). In contrast, at the same serum dilution only one (A1) of the six animals vaccinated with the unmodified envelope developed antibody responses able to neutralize SF162ΔV2 infection, and that by only 50%. The remaining five animals in this group failed to elicit antibody responses potent enough to neutralize SF162ΔV2-infection to any significant extent at this dilution. Differences in neutralizing potential between sera collected from animals immunized with the modified immunogen and those immunized with the unmodified immunogen were also evident when their ability to neutralize the SF 162 virus was compared (FIG. 7B). Sera collected from four (A8, A9, A10 and A12) out of six animals immunized with the modified antigen neutralized SF162-infection between 70% and 90% at 1:100 to 1:300 dilutions. In contrast, none of the sera collected from animals immunized with the unmodified antigen could inhibit SF162-infection by 70%-90% at the same dilutions. Generation of cross-reactive neutralizing antibodies in rabbits: The fact that the SF162ΔV2-derived envelope immunogen was capable of eliciting higher titers of neutralizing antibodies against the parental SF162 isolate (which expresses the full envelope) than the immunogen derived from the SF162 isolate itself, prompted us to examine whether the modified immunogen was also more effective in eliciting cross-reactive neutralizing antibodies, i.e., antibodies capable of neutralizing heterologous to the vaccine primary HIV-1 isolates. Several such isolates were tested whose neutralization susceptibility to various monoclonal antibodies was previously documented (D'Souza, M. P., D. Livnat, J. A. Bradac, and S. H. Bridges. 1997. Evaluation of monoclonal antibodies to human immunodeficiency virus type 1 primary isolates by neutralization assays: performance criteria for selecting candidate antibodies for clinical trials. AIDS Clinical Trials Group Antibody Selection Working Group. J. Infect. Dis. 175:1056-62). Only two (92US714 and the 92HT593) out of the six isolates, examined where neutralized by antibodies elicited by the unmodified immunogen (Table 1, below).

TABLE 1
Generation of cross-reactive neutralizing antibodies in rabbits

| | | ISOLATES | | | | | | | | | | | |
|---|---|---|---|---|---|---|---|---|---|---|---|---|---|
| | | 91US054 | | 92US657 | | 92US660 | | 92HT593 | | 91US056 | | 92US714 | |
| | Animals | (50) | (80) | (50) | (80) | (50) | (80) | (50) | (80) | (50) | (80) | (50) | (80) |
| Unmodified SF162gp140 | A1 | − | − | − | − | − | − | − | − | − | − | − | − |
| | A2 | − | − | − | − | − | − | + | − | − | − | + | − |
| | A3 | − | − | − | − | − | − | − | − | − | − | + | − |
| | A4 | − | − | − | − | − | − | − | − | − | − | + | − |
| | A5 | − | − | − | − | − | − | + | + | − | − | + | + |
| | A6 | − | − | − | − | − | − | − | − | − | − | + | − |
| Modified AV2gp140 | A7 | + | + | − | − | − | − | + | + | + | + | + | − |
| | A8 | + | + | − | − | − | − | + | + | + | + | + | + |
| | A9 | + | − | + | − | − | − | + | + | + | + | + | + |
| | A10 | − | − | + | + | − | − | + | + | − | − | + | + |
| | A11 | − | − | − | − | − | − | − | − | − | − | − | − |
| | A12 | − | − | − | − | − | − | − | − | − | − | − | − |

The neutralizing activity was evaluated at 1:10 dilution, taking into consideration the non-specific neutralization recorded with sera collected from animals vaccinated with the DNA vector alone (see Materials and Methods for details).
− 50% specific neutralization was not recorded.
+ 50% or 80% specific neutralization was recorded. Results are from three independent neutralization experiments.

With the exception of animal A1, all other animals developed neutralizing antibodies against 92US714, while only animals A2 and A5 generated neutralizing antibodies against 92HT593. In contrast, four out of the six animals immunized with the modified ΔV2gp120 immunogen generated cross-reactive neutralizing antibodies against most of the heterologous isolates tested. In addition, the neutralization potency of sera collected from animals immunized with the modified immunogen was higher than that of sera collected from animals immunized with the unmodified immunogen (see Table 1, above). Thus, although 80% inhibition of infection was frequently recorded with the former sera, this level of inhibition was recorded in only two instances (sera from animal A5 versus the 92US714 and 92HT593 isolates).

Development of antibodies in Rhesus macaques vaccinated with the modified ΔV2gp140 immunogen: The above results prompted an evaluation of the immunogenic potential of the unmodified SF162gp140 and modified ΔV2gp140 antigens in Rhesus macaques, an animal model where the protective potential of vaccine-elicited antibodies can eventually be evaluated. Macaques were vaccinated with these two immunogens using the DNA-prime followed by protein-boosting vaccination methodology.

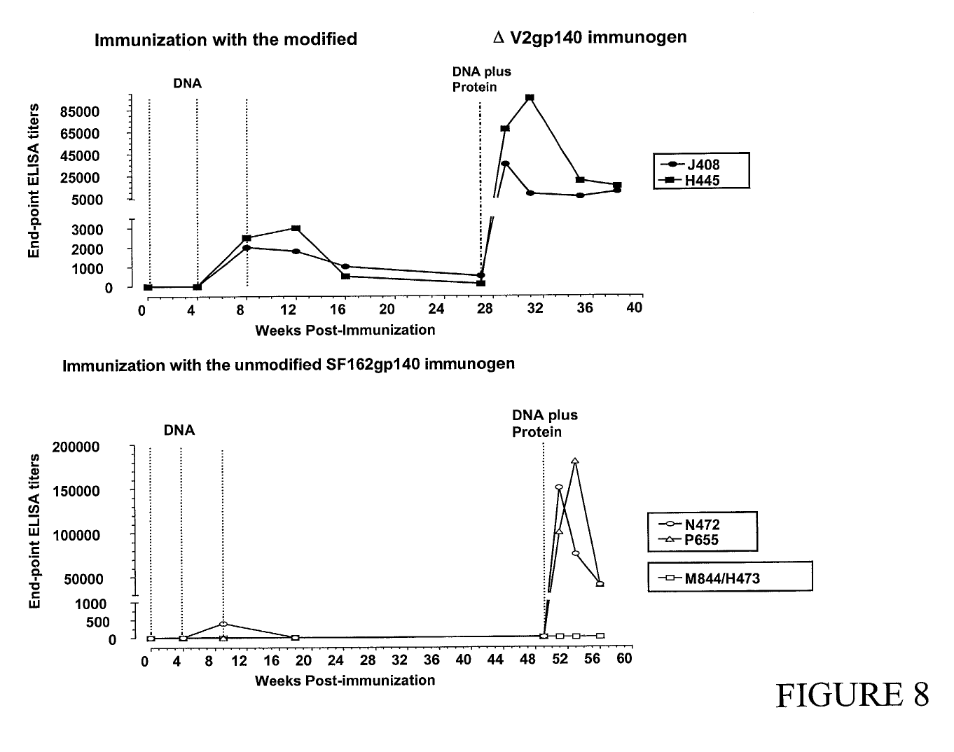

Envelope-specific antibodies became detectable following the second DNA immunization (FIG. 8). At this stage, end point ELISA titers in animals immunized with the modified antigen (animals J408 and H445) were in the order of 1:2,000. In contrast, in animals immunized with the unmodified envelope (animals N472 and P655), antibodies were only detectable in animal N472 (end point ELISA titers in the order of 1:500). With the exception of animal H445, the third DNA immunization did not further increase the antibody titers. Anti-gp120 and anti-gp41 antibodies were generated synchronously during DNA immunization.

During the subsequent five to ten months of observation, antibodies were undetectable in animals immuniz with the unmodified SF162gp140 immunogen, while in animals immunized with the modified ΔV2gp140 immunogen the antibodies were always detectable, but their titers declined over time.

Following the DNA plus protein ‘booster’ immunization, the antibody titers increased significantly in all animals. At their peak value (reached within 2-4 weeks post-‘boosting’), end-point ELISA antibody titers animals immunized with the modified ΔV2gp140 immunogen were 1:30,000 for animal J408 and 1:110,00 for animal H445. The titers decreased gradually over time and remained stable at approximately 1:8,000 for several weeks in both animals. Higher peak antibody titers were recorded in animals vaccinated with the unmodified SF162gp 140 immunogen (end-point ELISA antibody titers of 1:150,000 in animal N472 and 175,000 in animal P655). During the following 7 weeks of observation the antibody titers decreased more rapidly in both animals to approximately 1:35,000. Thus, in contrast to what was recorded in rabbits, in macaques the unmodified immunogen generated higher titers of binding antibodies than the modified immunogen.

As expected anti-HIV envelope antibodies were not generated in control animals (M844 and H473) immunized with the DNA vector alone.

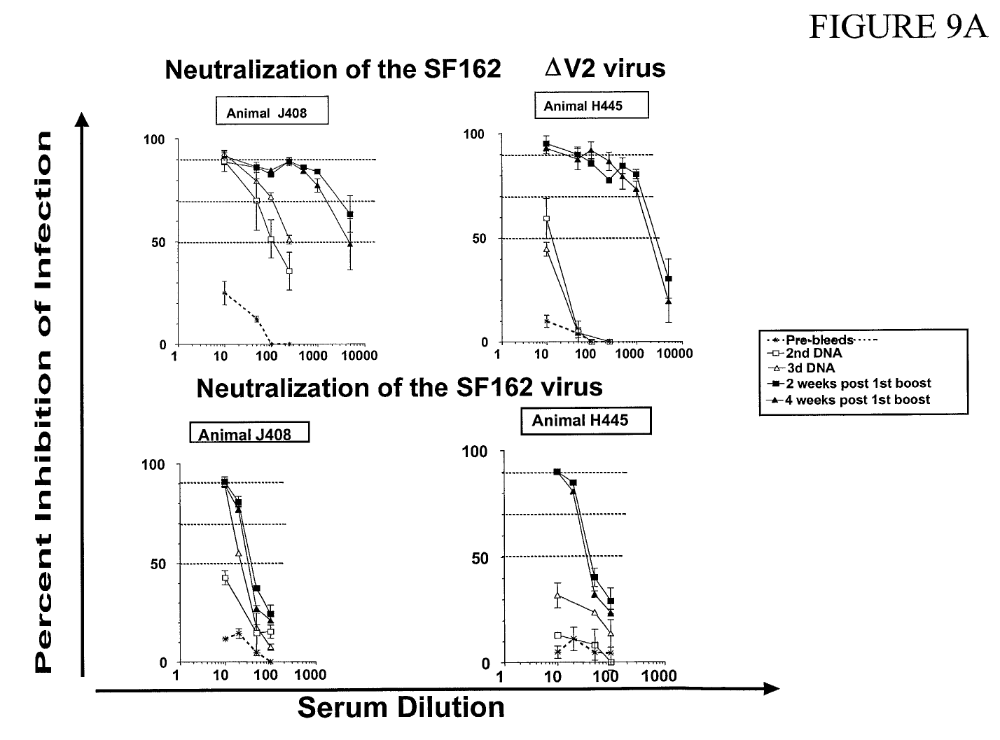

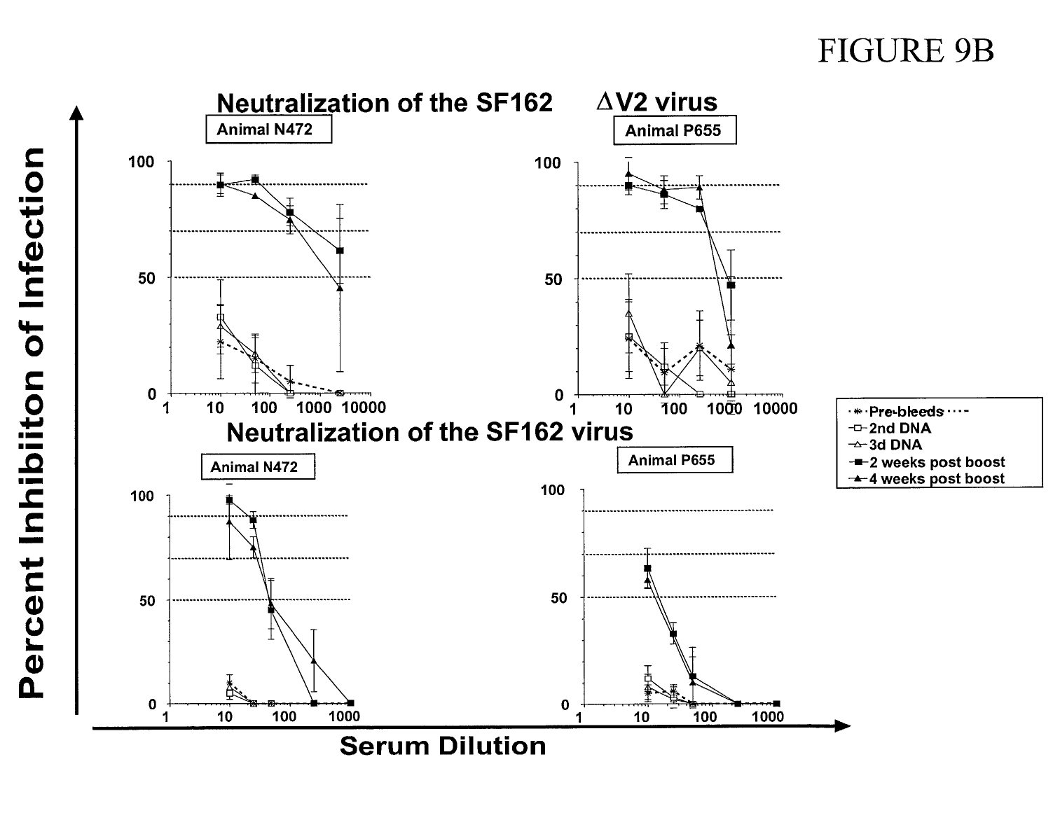

Neutralizing activity of macaque sera against the homologous SF162ΔV2 and parental SF162 isolates: During the DNA phase of immunization, only animals immunized with the modified ΔV2gp140 immunogen elicited neutralizing antibodies against the SF162 and SF162ΔV2 viruses (FIG. 9A-B). Following the second DNA immunization, animal J408 developed neutralizing antibodies against the homologous SF162ΔV2, but not the parental SF162, isolate (FIG. 9A). The titer of neutralizing antibodies in animal J408 increased following the third DNA immunization, at which point neutralization of both isolates was recorded, although the titers of binding antibodies did not increase in parallel (FIG. 9B). In contrast, much weaker neutralizing antibody responses against the SF162ΔV2 and no neutralizing responses against the SF162 virus were elicited in animal H445, even though this animal generated similar titers of binding antibodies to those generated in animal J408 (FIG. 9B).

Two weeks following the DNA plus protein ‘booster’ immunization sera collected from animals immunized with either immunogen inhibited SF162ΔV2-infection. The neutralization potency of sera collected from animals immunized with the modified immunogen was higher than that of sera collected from animals immunized with the unmodified immunogen. For example, 50% inhibition of SF162ΔV2-infection was recorded at dilutions of 1:2,000 to 1:5,000 from the former sera, but this level of inhibition was not recorded at this dilutions with sera collected from the latter sera. Both ΔV2gp140-immunized animals generated strong neutralizing antibodies against the parental SF162 virus, while only one (N472) of the two animals immunized with the SF162gp140 immunogen generated neutralizing antibodies against this virus. Changes in the neutralizing potency of these sera were not recorded during the subsequent two weeks, even tough changes in the antibody titer levels were detectable during this period (FIG. 9). Control animals (M844 and H473) vaccinated with the vector alone did not develop neutralizing antibodies.

Neutralization of heterologous primary HIV-1 isolates by macaques sera: The breath of the neutralizing antibody responses elicited in macaques immunized with the modified and unmodified immunogens, was evaluated by comparing the ability of sera collected from macaques immunized with these two immunogens to block infection of heterologous primary clade B HIV-1 isolates. The susceptibility of these isolates to neutralization by various MAbs was previously reported (D'Souza, M. P., D. Livnat, J. A. Bradac, and S. H. Bridges. 1997. Evaluation of monoclonal antibodies to human immunodeficiency virus type 1 primary isolates by neutralization assays: performance criteria for selecting candidate antibodies for clinical trials. AIDS Clinical Trials Group Antibody Selection Working Group. J. Infect. Dis. 175:1056-62). During the serum neutralization experiments, in parallel the susceptibility was evaluated of these isolates to neutralization by two of the most commonly used primary-isolate neutralizing MAbs (2F5 and 2G12) (Table 2).

TABLE 2
Neutralization of heterologous primary HIV-1 isolates by macaque sera

| | | | IMMUNOGEN | | | | | | | |
|---|---|---|---|---|---|---|---|---|---|---|
| | | | ΔV2GP140 | | | | SF162GP140 | | | |
| | Mab | | J408 | | H445 | | P655 | N472 | SF2gp120 | |
| ISOLATES | 2F5 | 2G12 | (A)^{&} | (B) | (A) | (B) | (A) | (A) | L714 | L814 |
| 91US056 | 60 | 70 | 90 | − | 65 | − | − | − | − | − |
| (R5) | | | | | | | | | | |
| 92US714 | 70 | 20 | 85 | − | 85 | − | − | − | − | − |
| (R5) | | | | | | | | | | |
| 92US660 | 75 | 70 | 50 | − | 80 | − | − | − | − | − |
| (R5) | | | | | | | | | | |
| 92HT593 | 75 | 80 | − | − | − | − | − | − | − | − |
| (R5X4) | | | | | | | | | | |
| 92US657 | NT | NT | − | − | − | 65 | − | − | − | − |
| (R5) | | | | | | | | | | |
| BZ167 | 90 | 75 | NT | − | NT | 80 | NT | NT | NT | NT |
| (R5X4) | | | | | | | | | | |
| ADA | NT | NT | 90 | 50 | 90 | 80 | NT | NT | NT | NT |
| (R5) | | | | | | | | | | |

Values represent the percent neutralization of a given HIV-1 isolate by sera (1:10 dilution) collected from animals immunized with the modified ΔV2gp140 (J408 and H445), unmodified SF162gp140 (P655 and N472) and recombinant gp120 (L714 and L814). The co-receptor usage of each isolate is shown in parenthesis.
The percent neutralization was calculated as described in Materials and Methods taking into consideration the non-specific neutralization recorded with sera collected from the same animals prior to the initiation of the immunization schedule.
^{&}(A) sera collected 2 weeks following the DNA plus protein ‘booster’ immunization of animals J408 and H445. Values represent averages from two to three independent experiments. The susceptibility of these isolates to neutralization by 2F5 and 2G12 at 25 μg/ml of MAb is also presented.
NT: Not evaluated.

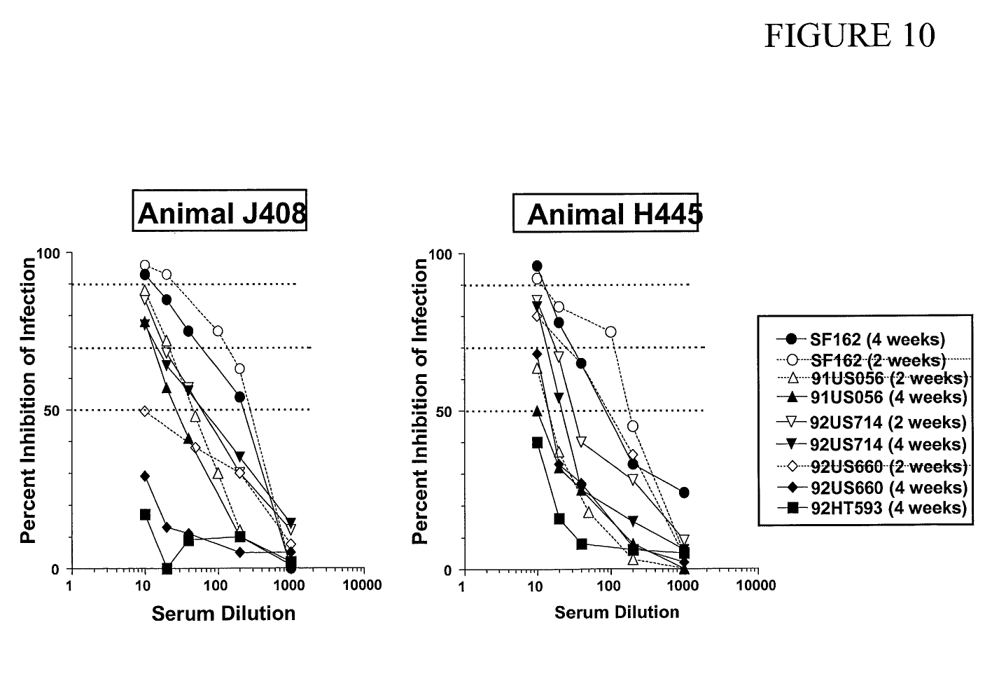

Heterologous isolate-neutralization was not recorded (less than 50% inhibition of infection at 1:10 serum dilution) during the DNA-phase of immunization in macaques. Two weeks following the DNA plus protein ‘booster’ immunization, sera collected from the two animals vaccinated with the modified ΔV2gp140 protein, neutralized some of the heterologous primary HIV-1 isolates tested (FIG. 10). At the lowest serum dilution tested (1:10), and when non-specific neutralization recorded with pre-immunization sera was taken into consideration (see Materials and Methods for details), 80-90% inhibition of infection was only recorded with the ADA, 91US056 and 92US714 isolates by J408 sera and with the ADA, 92US714 and 92US660 isolates with the H445 sera (FIG. 10 and Table 2). The cross-neutralizing activity of the sera collected from these two animals differed. For example, 92US660-infection was inhibited by 80% and 50%, by H445 and J408 sera, respectively. The serum cross-neutralizing activity decreased during the subsequent weeks of observation (FIG. 10). Sera collected 5 weeks following this DNA plus protein ‘booster’ immunization, had no cross-reactive neutralizing activity, even though potent neutralization of the SF162 and SF162ΔV2 isolates was still recorded.

Despite the fact that following this DNA plus protein ‘booster’ immunization, the binding antibody titers in animals vaccinated with the unmodified immunogen were higher than those in animals vaccinated with the modified immunogen (FIG. 8), the former sera failed to neutralize any of the heterologous isolates tested (Table 2) (i.e., less than 50% specific neutralization was recorded). Thus, although in rabbits the unmodified immunogen was able to elicit (albeit much less efficiently than the modified immunogen) neutralizing antibodies against some heterologous primary HIV-1 isolates (Table 1), it failed to do so in rhesus macaques.

In parallel, the susceptibility was evaluated of the heterologous isolates to neutralization by sera collected from macaques that have been immunized with the recombinant SF2-derived gp120 protein. This protein was previously evaluated as a vaccine candidate and was ineffective in eliciting cross-reactive neutralizing antibodies, i.e., less than 50% neutralization at serum dilutions of 1:10 was recorded (Mascola, J. R., S. W. Snyder, O. S. Weislow, S. M. Belay, R. B. Belshe, D. H. Schwartz, M. L. Clements, R. Dolin, B. S. Graham, G. J. Gorse, M. C. Keefer, M. J. McElrath, M. C. Walker, K. F. Wagner, J. G. McNeil, F. E. McCutchan, D. S. Burke and the NIAID AIDS vaccine evaluation group. 1996. Immunization with envelope subunit vaccine products elicits neutralizing antibodies against laboratory-adapted but not primary isolates of human immunodeficiency virus type 1. J. Infect. Dis. 173:340-348). All the isolates tested here were not susceptible to neutralization by antibodies elicited by the SF2 gp120 protein (Table 2).

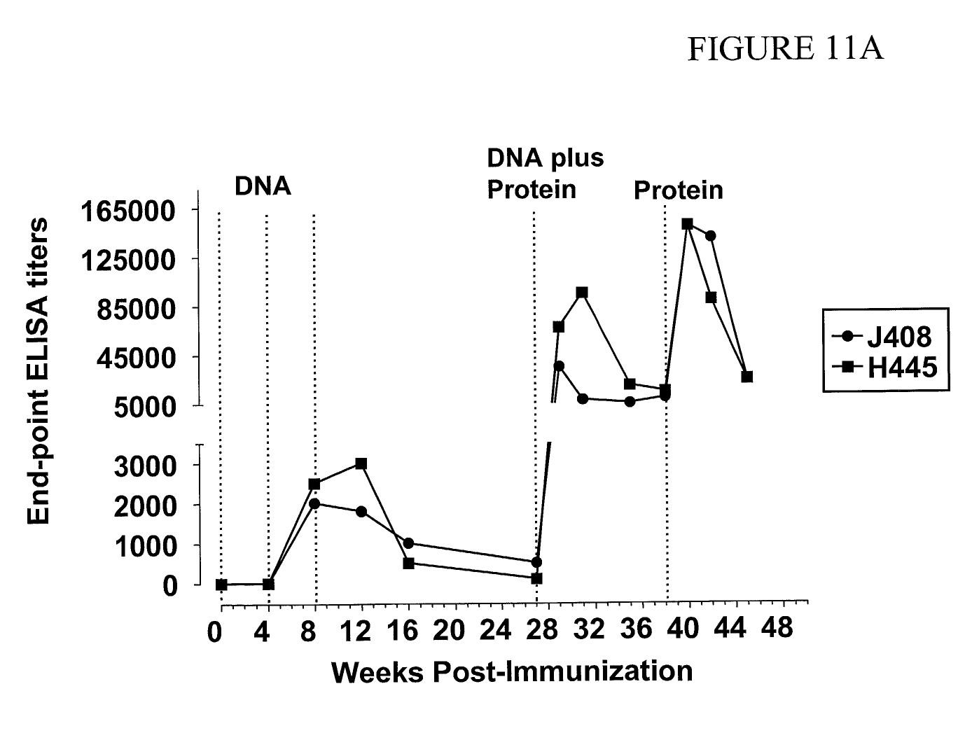

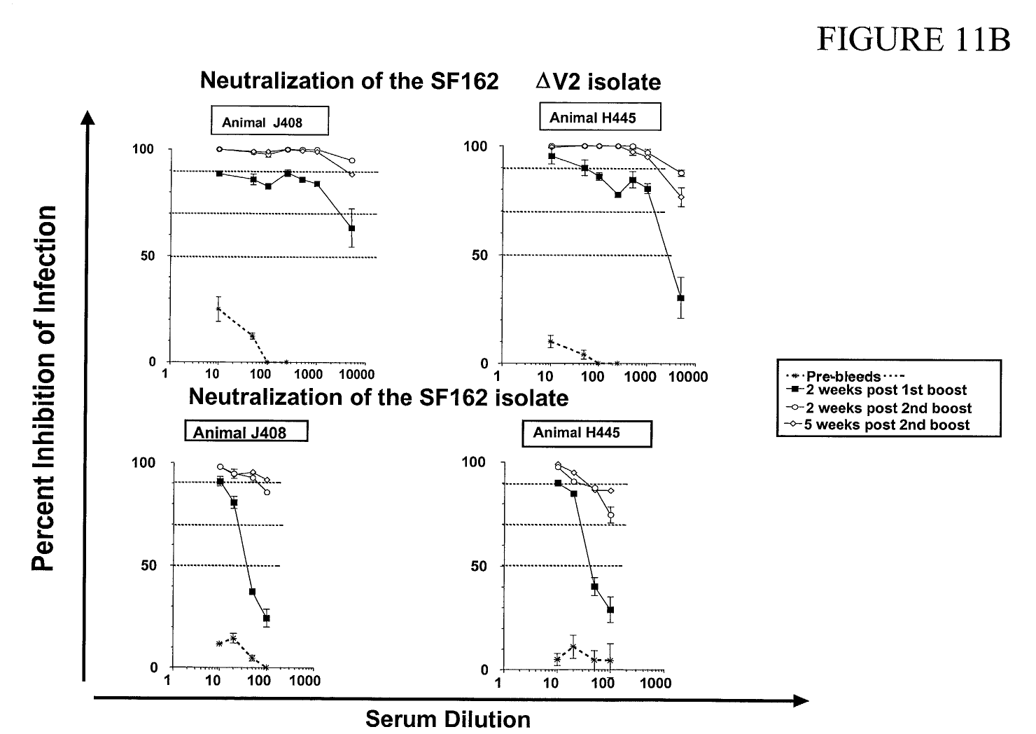

Second ‘booster’ immunization with the modified ΔV2gp140 protein: Although the above results indicated that the modified ΔV2gp140 immunogen was indeed more effective in eliciting cross-reactive neutralizing antibody responses than the unmodified immunogen, these responses were weaker than those recorded against the parental SF162 isolate (FIG. 11A-B). In an effort to further increase the potency and breath of these responses, an attempt was made to further ‘boost’ the antibody titers in animals H445 and J408 by immunizing them one additional time with the purified oligomeric ΔV2gp140 protein (this time in the absence of DNA-immunization).

An increase in antibody-titers was indeed recorded following this protein ‘boost’, so that at their peak valu (1:145,000 end-point ELISA titers) the titers were approximately 3 fold higher than those recorded during first ‘booster’ immunization with DNA plus protein (FIG. 11A). In parallel, a significant increase was found in the titer of neutralizing antibodies against the homologous SF162ΔV2 and parental SF162 isolate: (FIG. 11B). No differences in the neutralizing potential of the sera collected 2 and 5 weeks following thi last ‘boost’ were recorded, even though the binding antibody titers decreased significantly during the same period. Unexpectedly, however, the neutralizing potential of the same sera against most of the heterologou primary isolates tested generally decreased (Table 2). Thus, with the exception of the BZ167, 92US657 and ADA isolates, all the heterologous isolates tested were resistant to neutralization by sera collected 2 weeks following the second ‘boost’. Interestingly, although isolate 92US657 was resistant to neutralization by sei collected following the first boost, it became susceptible to neutralization by sera collected following the second boost.

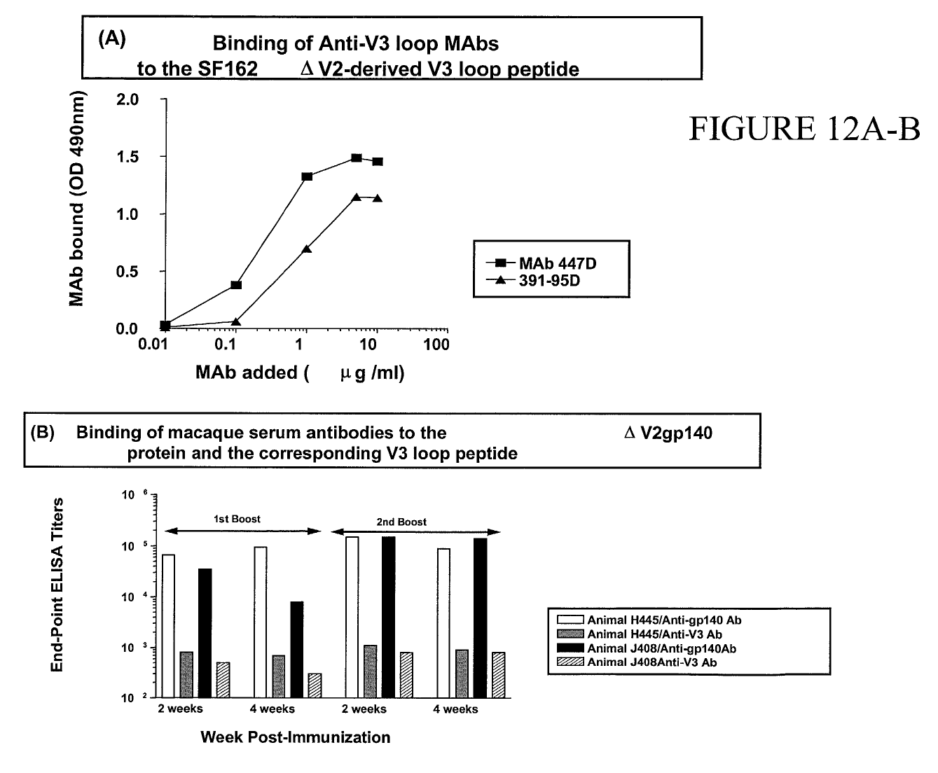

Generation of anti-V3 loop antibodies in Rhesus vaccinated with the modified ΔV2gp140 immunogen: One explanation for the increase in neutralizing activity against the parental SF162 and homologous SF162ΔV2 viruses and the decrease in neutralizing activity against the heterologous isolates following the second ‘booster’ immunization, is that multiple immunizations with the modified ΔV2gp140 protein increased the titer of antibodies directed against epitopes that are uniquely (or predominantly) expressed on the SF162 and SF162ΔV2 envelopes. It is conceivable that multiple immunizations with the ΔV2gp140 protein result in the generation of high titers of anti-V3 loop antibodies. To determine the titer of such antibodies, a V3 loop peptide-based ELISA assays was used using the SF162/SF162ΔV2-derived V3 loop (FIG. 12A-B). This peptide was recognized by antibodies binding to both linear (447D) (Conley, A. J., M. K. Gorny, J. A. Kessler, second, L. J. Boots, M. Ossorio-Castro, S. Koenig, D. W. Lineberger, E. A. Emini, C. Williams, and S. Zolla-Pazner. 1994. Neutralization of primary human immunodeficiency virus type 1 isolates by the broadly reactive anti-V3 monoclonal antibody, 447-52D. J. Virol. 68:6994-7000; Gomy, M. K., A. J. Conley, S. Karwowska, A. Buchbinder, J.-Y. Xu, E. A. Emini, S. Koenig, and S. Zolla-Pazner. 1992. Neutralization of diverse human immunodeficiency virus type 1 variants by an anti-V3 human monoclonal antibody. J. Virol. 66:7538-7542) and conformational (391-95D) (Seligman, S. J., J. M. Binley, M. K. Gomy, D. R. Burton, S. Zolla-Pazner, and K. A. Sokolowski. 1996. Characterization by serial competition ELISAs of HIV-1 V3 loop epitopes recognized by monoclonal antibodies. Mol. Immunol. 33:737-745) epitopes (FIG. 11A). Although anti-V3 loop antibodies were generated upon immunization of macaques with the modified ΔV2gp140 immunogen, their titers were much lower than those against the entire envelope (FIG. 11B). In addition, the second ‘booster’ immunization did not increase the titer of anti-V3 loop antibodies. It should be noted, however, that certain anti-V3 loop antibodies present in the serum of these animals may not interact efficiently with the V3 loop peptide in an ELISA format, while they may bind to their epitopes on the native envelope (Moore, J. P. 1993. The reactivities of HIV-1 +human sera with solid-phase V3 loop peptides can be poor predictors of their reactivities with V3 loops on native gp120 molecules. AIDS Res. Hum. Retroviruses 9:209-19). Additionally, the V3 loop peptide used here does not span the carboxy and amino termini of the V3 loop and the assay does not detect antibodies targeting these two regions. Thus, a more detailed examination of the epitope-specificity of the antibodies elicited by the modified ΔV2gp140 immunogen is required.

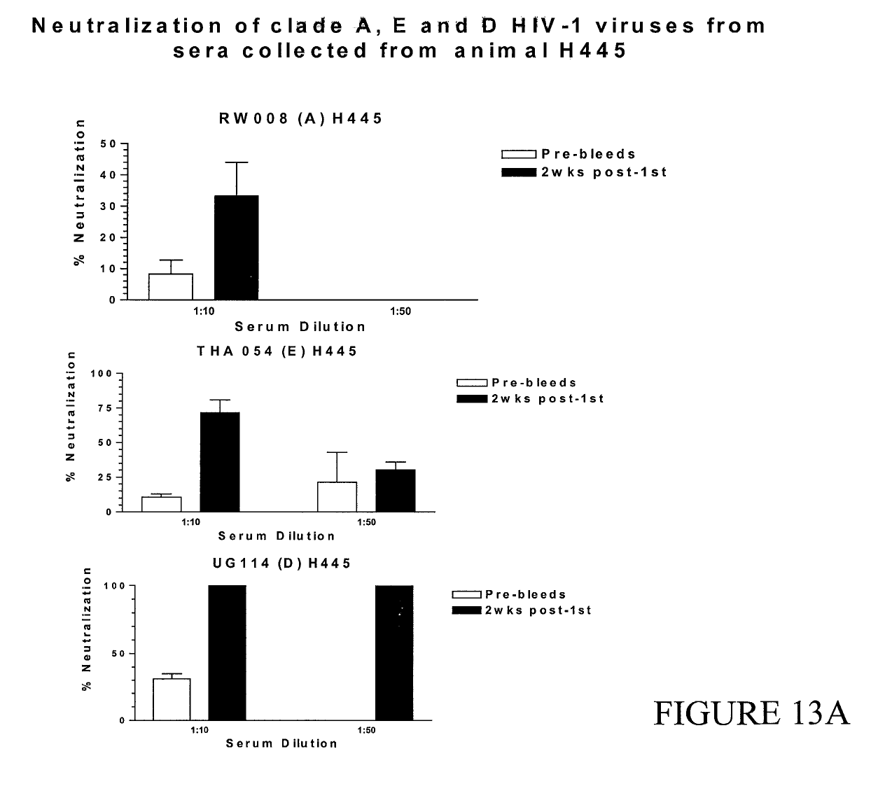

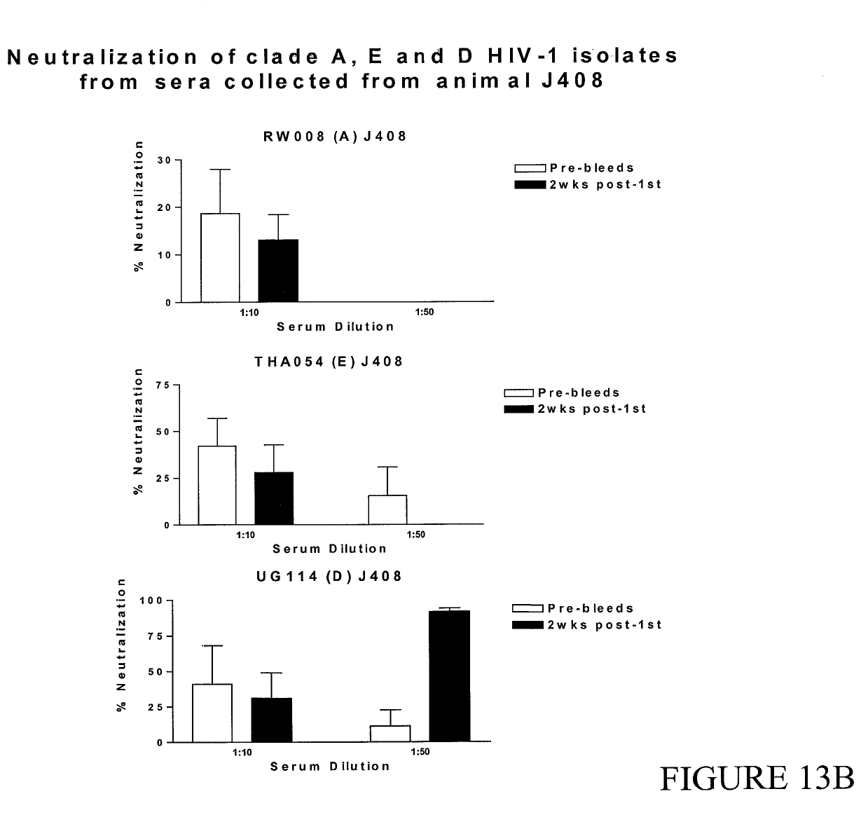

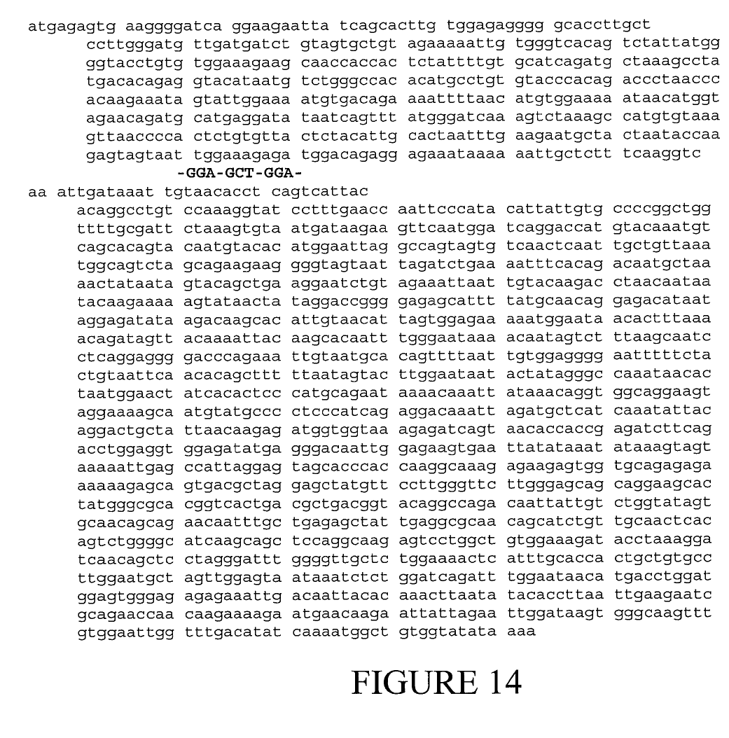

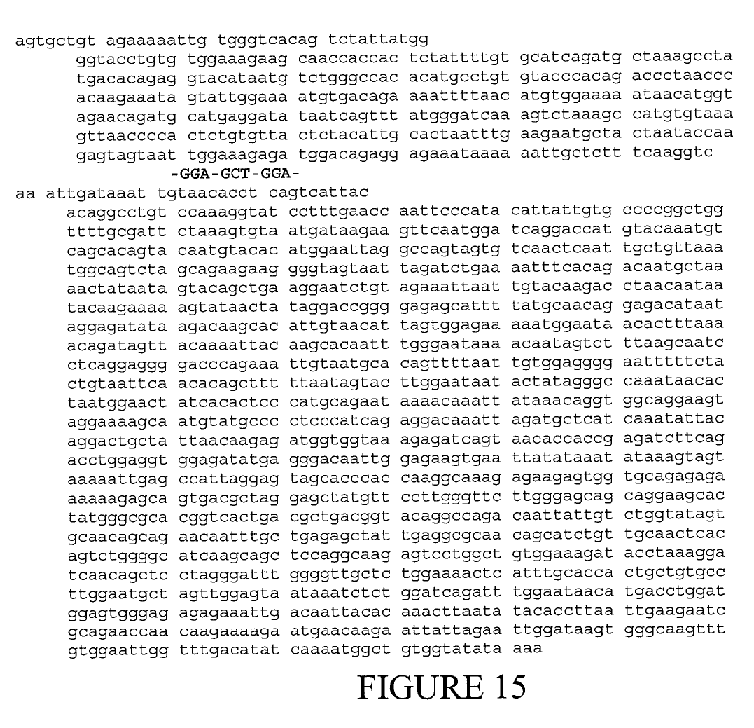

FIGS. 13A and B demonstrate the heterologous immune response elicited by the immunogens of the invention, by the neutralization of HIV-1 viruses of different clades. FIG. 13A shows the neutralization using serum from animals H445; FIG. 13B using serum from animal J408.

While the invention has been described and illustrated herein by references to the specific embodiments, various specific material, procedures and examples, it is understood that the invention is not restricted to the particular material combinations of material, and procedures selected for that purpose. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

What is claimed is:

1. A method for immunizing an animal against heterologous HIV-1 comprising administering to said animal an immunogen comprising at least one modified HIV-1 envelope protein or fragment thereof, or DNA or virus encoding said at least one modified HIV-1 envelope protein or fragment thereof, or a combination thereof, said modified envelope protein or fragment thereof having a V2 region deletion, wherein said animal exhibits immunity to at least one HIV-1 strain other than that of said immunogen.

2. The method of claim 1 wherein said immunity comprises a humoral response.

3. The method of claim 1 wherein said immunogen comprises a modified HIV-1 envelope protein from a clade-B HIV-1 strain.

4. The method of claim 3 wherein said HIV-strain is SF162.

5. The method of claim 4 wherein said modified HIV-1 envelope protein is SEQ ID No:2 or SEQ ID No:4.

5. The method of claim 4 wherein said DNA encoding said at least one modified HIV-1 envelope protein is SEQ ID No:1 or SEQ ID No:3.

6. The method of claim 2 wherein said humoral response comprises neutralizing antibodies.

7. The method of claim 2 wherein said humoral response comprises protective antibodies.

8. The method of claim 1 wherein said animal is a human.

9. A method for eliciting a heterologous immune response to HIV-1 in an animal comprising immunizing said animal with an immunogen comprising at least one modified HIV-1 envelope protein or fragment thereof, or DNA or virus encoding said at least one modified HIV-1 envelope protein or fragment thereof, or a combination thereof, said modified envelope protein or fragment thereof having a V2 region deletion, wherein said animal exhibits a an envelope-specific immune response to at least one HIV-1 strain other than that of said immunogen.

10. The method of claim 9 wherein said envelope-specific immune response comprises a humoral response.

11. The method of claim 9 wherein said immunogen comprises a modified HIV-1 envelope protein from a clade-B HIV-1 strain.

12. The method of claim 11 wherein said HIV-strain is SF162.

13. The method of claim 12 wherein said modified HIV-1 envelope protein is SEQ ID No:2 or SEQ ID No:4.

14. The method of claim 12 wherein said DNA encoding said at least one modified HIV-1 envelope protein is SEQ ID No:1 or SEQ ID No:3.

15. The method of claim 10 wherein said humoral response comprises neutralizing antibodies.

16. The method of claim 10 wherein said humoral response comprises protective antibodies.

17. The method of claim 9 wherein said animal is a human.

18. A pharmaceutical composition for immunizing an animal against HIV-1 virus comprising an effective heterologous envelope-specific immune response-eliciting amount of at least one modified HIV-1 envelope protein or fragment thereof, or DNA or virus encoding said at least one modified HIV-1 envelope protein or fragment thereof, or a combination thereof, said modified envelope protein or fragment thereof having a V2 region deletion; and a pharmaceutically-acceptable carrier or excipient.

19. The pharmaceutical composition of claim 18 wherein said modified HIV-1 envelope protein is from a clade-B HIV-1 strain.

20. The pharmaceutical composition of claim 19 wherein said HIV-1 strain is SF162.

21. The pharmaceutical composition of claim 20 wherein said modified HIV-1 envelope protein is SEQ ID No:2 or SEQ ID No:4.

22. The pharmaceutical composition of claim 20 wherein said DNA encoding said at least one modified HIV-1 envelope protein is SEQ ID No:1 or SEQ ID No:3.

23. A method for assessing whether a compound is capable of generating protective antibodies in an animal against at least one heterologous strain of HIV-1, said animal capable of developing protective antibodies against wild-type HIV-1, said method comprising the steps of immunizing said animal with said compound, depleting said animal of its CD8+ T-lymphocytes, and assessing the presence of protective antibodies in the said animal to at least one heterologous strain of HIV-1.

24. The method of claim 23 wherein said depleting is carried out by administering to said animal anti-CD8 monoclonal antibodies.

25. The method of claim 23 wherein said compound is an HIV-derived polypeptide or fragment thereof or a DNA or virus encoding said peptide or fragment thereof.

26. The method of claim 23 wherein said immunizing is carried out with a DNA vaccine, a protein, or a combination thereof.

27. The method of claim 23 wherein said neutralizing antibodies are protective antibodies.


SEQUENCE LISTING (4 sequences)

SEQ ID NO: 1 - DNA, 1941 bp, Human immunodeficiency virus type 1
atgagagtgaaggggatcaggaagaattatcagcacttgtggagagggggcaccttgctccttgggatgttgatgatctgtagtgctgtagaaaaattgtgggtcacagtctattatggggtacctgtgtggaaagaagcaaccaccactctattttgtgcatcagatgctaaagcctatgacacagaggtacataatgtctgggccacacatgcctgtgtacccacagaccctaacccacaagaaatagtattggaaaatgtgacagaaaattttaacatgtggaaaaataacatggtagaacagatgcatgaggatataatcagtttatgggatcaaagtctaaagccatgtgtaaagttaaccccactctgtgttactctacattgcactaatttgaagaatgctactaataccaagagtagtaattggaaagagatggacagaggagaaataaaaaattgctctttcaaggtcggagctggaaaattgataaattgtaacacctcagtcattacacaggcctgtccaaaggtatcctttgaaccaattcccatacattattgtgccccggctggttttgcgattctaaagtgtaatgataagaagttcaatggatcaggaccatgtacaaatgtcagcacagtacaatgtacacatggaattaggccagtagtgtcaactcaattgctgttaaatggcagtctagcagaagaaggggtagtaattagatctgaaaatttcacagacaatgctaaaactataatagtacagctgaaggaatctgtagaaattaattgtacaagacctaacaataatacaagaaaaagtataactataggaccggggagagcattttatgcaacaggagacataataggagatataagacaagcacattgtaacattagtggagaaaaatggaataacactttaaaacagatagttacaaaattacaagcacaatttgggaataaaacaatagtctttaagcaatcctcaggaggggacccagaaattgtaatgcacagttttaattgtggaggggaatttttctactgtaattcaacacagctttttaatagtacttggaataatactatagggccaaataacactaatggaactatcacactcccatgcagaataaaacaaattataaacaggtggcaggaagtaggaaaagcaatgtatgcccctcccatcagaggacaaattagatgctcatcaaatattacaggactgctattaacaagagatggtggtaaagagatcagtaacaccaccgagatcttcagacctggaggtggagatatgagggacaattggagaagtgaattatataaatataaagtagtaaaaattgagccattaggagtagcacccaccaaggcaaagagaagagtggtgcagagagaaaaaagagcagtgacgctaggagctatgttccttgggttcttgggagcagcaggaagcactatgggcgcacggtcactgacgctgacggtacaggccagacaattattgtctggtatagtgcaacagcagaacaatttgctgagagctattgaggcgcaacagcatctgttgcaactcacagtctggggcatcaagcagctccaggcaagagtcctggctgtggaaagatacctaaaggatcaacagctcctagggatttggggttgctctggaaaactcatttgcaccactgctgtgccttggaatgctagttggagtaataaatctctggatcagatttggaataacatgacctggatggagtgggagagagaaattgacaattacacaaacttaatatacaccttaattgaagaatcgcagaaccaacaagaaaagaatgaacaagaattattagaattggataagtgggcaagtttgtggaattggtttgacatatcaaaatggctgtggtatataaaa

SEQ ID NO: 2 - protein, 646 aa, Human immunodeficiency virus type 1
MRVKGIRKNYQHLWRGGTLLLGMLMICSAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVGAGKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAKRRVVQREKRAVTLGAMFLGFLGAAGSTMGARSLTLTVQARQLLSGIVQQQNNLLRAIEAQQHLLQLTVWGIKLQARVLAVERYLKDQQLLGIWGCSGKLICTTAVPWNASWSNKSLDQIWNNMTWMEWEREIDNYTNLIYTLIEESQNQQEKNEQELLELDKWASLWNWFDISKWLWYIK

SEQ ID NO: 3 - DNA, 1860 bp, Human immunodeficiency virus type 1
agtgctgtagaaaaattgtgggtcacagtctattatggggtacctgtgtggaaagaagcaaccaccactctattttgtgcatcagatgctaaagcctatgacacagaggtacataatgtctgggccacacatgcctgtgtacccacagaccctaacccacaagaaatagtattggaaaatgtgacagaaaattttaacatgtggaaaaataacatggtagaacagatgcatgaggatataatcagtttatgggatcaaagtctaaagccatgtgtaaagttaaccccactctgtgttactctacattgcactaatttgaagaatgctactaataccaagagtagtaattggaaagagatggacagaggagaaataaaaaattgctctttcaaggtcggagctggaaaattgataaattgtaacacctcagtcattacacaggcctgtccaaaggtatcctttgaaccaattcccatacattattgtgccccggctggttttgcgattctaaagtgtaatgataagaagttcaatggatcaggaccatgtacaaatgtcagcacagtacaatgtacacatggaattaggccagtagtgtcaactcaattgctgttaaatggcagtctagcagaagaaggggtagtaattagatctgaaaatttcacagacaatgctaaaactataatagtacagctgaaggaatctgtagaaattaattgtacaagacctaacaataatacaagaaaaagtataactataggaccggggagagcattttatgcaacaggagacataataggagatataagacaagcacattgtaacattagtggagaaaaatggaataacactttaaaacagatagttacaaaattacaagcacaatttgggaataaaacaatagtctttaagcaatcctcaggaggggacccagaaattgtaatgcacagttttaattgtggaggggaatttttctactgtaattcaacacagctttttaatagtacttggaataatactatagggccaaataacactaatggaactatcacactcccatgcagaataaaacaaattataaacaggtggcaggaagtaggaaaagcaatgtatgcccctcccatcagaggacaaattagatgctcatcaaatattacaggactgctattaacaagagatggtggtaaagagatcagtaacaccaccgagatcttcagacctggaggtggagatatgagggacaattggagaagtgaattatataaatataaagtagtaaaaattgagccattaggagtagcacccaccaaggcaaagagaagagtggtgcagagagaaaaaagagcagtgacgctaggagctatgttccttgggttcttgggagcagcaggaagcactatgggcgcacggtcactgacgctgacggtacaggccagacaattattgtctggtatagtgcaacagcagaacaatttgctgagagctattgaggcgcaacagcatctgttgcaactcacagtctggggcatcaagcagctccaggcaagagtcctggctgtggaaagatacctaaaggatcaacagctcctagggatttggggttgctctggaaaactcatttgcaccactgctgtgccttggaatgctagttggagtaataaatctctggatcagatttggaataacatgacctggatggagtgggagagagaaattgacaattacacaaacttaatatacaccttaattgaagaatcgcagaaccaacaagaaaagaatgaacaagaattattagaattggataagtgggcaagtttgtggaattggtttgacatatcaaaatggctgtggtatataaaa

SEQ ID NO: 4 - protein, 619 aa, Human immunodeficiency virus type 1
SAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVGAGKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAKRRVVQREKRAVTLGAMFLGFLGAAGSTMGARSLTLTVQARQLLSGIVQQQNNLLRAIEAQQHLLQLTVWGIKLQARVLAVERYLKDQQLLGIWGCSGKLICTTAVPWNASWSNKSLDQIWNNMTWMEWEREIDNYTNLIYTLIEESQNQQEKNEQELLELDKWASLWNWFDISKWLWYIK